(19) United States
(12) Patent Application Publication
(10) Pub. No.: US 2016/0003843 A1
(43) Pub. Date: Jan. 7, 2016

(54) ENGINEERED ANTIBODY SCAFFOLDS

(71) Applicant: THE REGENTS OF THE UNIVERSITY OF CALIFORNIA, Oakland, CA (US)

(72) Inventors: James A. WELLS, Burlingame, CA (US); James T. KOERBER, San Francisco, CA (US); William DEGRADO, San Francisco, CA (US)

(21) Appl. No.: 14/776,122

(22) PCT Filed: Mar. 14, 2014
(86) PCT No.: PCT/US2014/027588

§ 371 Date: Sep. 14, 2015

Related U.S. Application Data
(60) Provisional application No. 61/788,343, filed on Mar. 15, 2013.

(51) Int. Cl. G01N 33/68

(52) U.S. Cl. CPC G01N 33/6854

(57) ABSTRACT

The invention described herein features methods and compositions for generating an antibody specific to post-translational modifications (PTMs). The methods and compositions provide a renewable synthetic antibody strategy that installs a novel motif-specific hot spot into an antibody scaffold that functions independently of the surrounding scaffold. Such antibodies provide a rapid and robust development of antibodies for signaling, diagnostic, and therapeutic applications.

### CROSS REFERENCE TO RELATED APPLICATIONS

The present application claims priority to U.S. Provisional Application No. 61/788,343, filed on Mar. 15, 2013, and which is incorporated herein by reference in its entirety for all purposes.

### STATEMENT OF FEDERALLY-SPONSORED RESEARCH

This invention was made with government support under Grant No. CA154802 awarded by the National Institutes of Health and Grant No. A12258 awarded by the Life Science Research Foundation. The government has certain rights in the invention.

### BACKGROUND OF THE INVENTION

Post-translational modifications (PTMs), such as phosphorylation, acetylation, sulfation, S-nitrosylation, methylation, glycosylation and proteolysis, play essential roles in modulating protein function throughout biology. In particular, phosphorylation is one of the most common regulatory mechanisms in eukaryotes, where roughly 20-30% of all proteins can be phosphorylated by over 500 kinases (1). Given the ubiquitous role of phosphorylation in signal transduction, it is not surprising that aberrant phosphorylation either directly causes or is a consequence of many human diseases, such as cancer and neurodegenerative disorders (2). Recent advances in phosphoproteomic methods have greatly expanded the number of known phosphorylation sites (>170,000) and identified global phosphorylation changes that occur during disease (3-6). Ultimately, the validation of key phosphorylation and other post-translational modification events is best conducted at the single-cell level where recent studies utilizing monoclonal antibodies (Abs) specific to particular post-translational modifications have elucidated how stochastic fluctuations and signaling cross-talk contribute to the overall cellular state (7, 8).

Detection using PTM reagents is limited to antibodies generated through the immunization of animals (9). However, the generation of a polyclonal PTM antibody is often imprecise, low-throughput, expensive, time-consuming, and not renewable. Furthermore, the development of monoclonal antibodies requires additional screening of numerous hybridomas to identify the Ab of interest, which is made more challenging by the rarity of Ab clones specific to post-translational modifications, which is estimated to be 0.1-5% (10, 11). Additionally, the lack of available sequences of PTM Abs generated by immunization makes structure-guided improvements to their biophysical properties (e.g. affinity or stability) extremely difficult.

Recent attempts to generate renewable phospho-specific Abs using in vitro selection methods, such as phage display (12-16), yeast display (17), and ribosome display (18), have been even less efficient than immunization methods (17, 19-22). Overall, both immunization and in vitro methods fail to generate high affinity Abs due to the fact that most of the naïve Abs do not possess any initial affinity for the small peptide antigens. Finally, disproportionately more phosphotyrosine (pTyr)-specific Abs exist than phosphoserine (pSer)- or phosphothreonine (pThr)-specific Abs, which is likely due to the larger surface area of pTyr. This fact has hindered the study of phosphorylation of serine and threonine phosphorylation, which account for 90% and 10% of all phosphorylation sites, respectively, compared to <0.05% for tyrosine (23). As an alternative to Abs, several groups have engineered PS reagents using endogenous phosphopeptide-binding domains such as Src-homology-2 (SH2) or forkhead-associated (FHA) domains, but the general utility of such non-antibody scaffolds has not been demonstrated and is limited by poor thermostabilities, weak affinities, and short epitopes recognized by such domains (24-26).

Very few commercially available antibodies are suitable for rapid, robust methods to generate high-quality, renewable, monoclonal post-translational modification detection reagents as the number of functionally important post-translational modification sites increases. The invention described herein solves these and other problems by providing a novel structure-guided Ab generation strategy that uses antibody scaffolds with engineered hot spots tailored to specific sequence motifs relating to particular post-translational modifications of interest.

### BRIEF SUMMARY OF THE INVENTION

Provided herein are high affinity Abs using a novel structure-guided Ab generation strategy that employs Ab scaffolds with engineered or identified hot spots tailored to bind to a sequence motif specific to a particular post-translational modification. In an embodiment, a method of identifying antibodies that specifically bind a protein comprising a post translational modification are provided. The method comprises (1) identifying a noncovalent post translational modification-binding motif in a protein; (2) inspecting known antibody CDRs for an anchoring pocket sequence that adopts the conformation of the noncovalent post translational modification-binding motif; (3) preparing an antibody scaffold comprising a CDR, wherein amino acid residues from the CDR comprise the anchoring pocket; (4) preparing a library comprising the antibody scaffold, wherein the CDR is randomized outside of the anchoring pocket; and (5) identifying antibodies in the library that specifically bind to a protein comprising a post translational modification.

In an exemplary embodiment, the antibody binds a specific protein comprising the post translational modification. In an exemplary embodiment, the antibody binds a specific post translational modification. In an exemplary embodiment, the post translational modification is an anion. In an exemplary embodiment, the post translational modification is phosphorylation, sulfation, acetylation, S-nitrosylation, methylation, proteolysis, or glycosylation. In an exemplary embodiment, the noncovalent post translational modification-binding motif recognizes an anion. In an exemplary embodiment, the noncovalent post translational modification-binding motif recognizes a phosphate, a sulfate, an acetyl, a methyl, a nitric oxide, a N-terminal alpha amine, a C-terminal carboxylate, a GalNAc sugar or a GlcNAc sugar. In an exemplary embodiment, the method further comprises the step of characterizing the anchoring pocket. In an exemplary embodiment, amino acid residues from two or more CDRs comprise the anchoring pocket.

In an embodiment, a method of identifying antibodies that specifically bind a protein comprising a post translational modification is provided. The method comprises (1) identifying a noncovalent post translational modification-binding motif in a protein; (2) engineering an antibody scaffold comprising a CDR, wherein amino acid residues from the CDR comprise an anchoring pocket that adopts the conformation of the noncovalent post translational modification-binding motif; (3) preparing a library comprising the antibody scaffold, wherein the CDR is randomized outside of the anchoring pocket; and (4) identifying antibodies in the library that specifically bind to a protein comprising a post translational modification.

In an exemplary embodiment, the antibody binds a specific protein comprising the post translational modification. In an exemplary embodiment, the antibody binds a specific post translational modification. In an exemplary embodiment, the post translational modification is an anion. In an exemplary embodiment, the post translational modification is phosphorylation, sulfation, acetylation, S-nitrosylation, methylation, proteolysis, or glycosylation. In an exemplary embodiment, the noncovalent post translational modification-binding motif recognizes an anion. In an exemplary embodiment, the noncovalent post translational modification-binding motif recognizes a phosphate, a sulfate, an acetyl, a methyl, a nitric oxide, a N-terminal alpha amine, a C-terminal carboxylate, a GalNAc sugar or a GlcNAc sugar. In an exemplary embodiment, the method further comprises the step of characterizing the anchoring pocket. In an exemplary embodiment, amino acid residues from two or more CDRs comprise the anchoring pocket.

### BRIEF DESCRIPTION OF THE DRAWINGS

FIG. 1 illustrates the design of phospho-specific antibody scaffold. (a) Structure of CDR H2 loop from antibody (PDB ID 1i8i) bound to aspartate in peptide antigen (31). Each H2 residue contributes to anchoring the peptide near the pocket (52_{H} and 52A_{H}), specificity (53_{H}, 55_{H}, and 56_{H}), or conformation (53_{H}). (b) Competition phage ELISAs with humanized Fab. Eight different mutant peptides containing D, A, S, T, Y, pS, pT, or pY at position 8 of the peptide were used as soluble competitors to inhibit Fab binding to the immobilized wild-type peptide (KGNYVVTDH) (n=3, error bars represent standard deviation). (c) Representative pooled phage ELISAs from selection of H2-targeted library against pSer peptide. After three rounds of selection, all library pools exhibited higher binding signal to the pSer peptide than the parent Fab (dashed line).

FIG. 2 illustrates the structure of nest motif in non-antibody and antibody scaffolds. (a) Nest motif present in barnase (61), in which three consecutive main-chain amides contact the carbonyl group from a different residue. (b) Structural alignment of CDR H2 bound to Asp/Glu. Alignment of CDR H2 region (50_{H}-56_{H}) from PDB ID 1i8i (62), 1frg (63), 2igf (64), 2qhr (65), 1dqj (66), 2nyy (67), 3bn9 (68), and 3ffd (69). All the antibodies contain G54_{H} (indicated by arrow) and make at least two hydrogen bonds between the Asp/Glu antigen residue and main-chain NH groups. (c) Alignment of the same CDR H2 region bound to sulfate ions from PDB ID 1seq (70), 2gsg (71), and 3vg0 (72).

FIG. 3 illustrates the selection and characterization of pSer-, pSer/pThr-, and pTyr-specific scaffolds. Competition ELISAs were used to determine the specificity of each antibody scaffold. For both pSAb (a) and pSTAb (b), no binding inhibition was observed for the unphosphorylated peptides up to 2 μM, whereas strong inhibition was observed for the phosphorylated peptides. For pYAb (c), weak inhibition was observed at high concentrations of the unphosphorylated Tyr peptide, but ˜20-fold less pTyr peptide was required to observe the same level of inhibition. The sequence logos of the antibody pools from which each lead clone was derived are depicted in the bottom panels. GS and H2 indicate the sequence logos from GS and H2 libraries selected against pSer and pThr. All clones that bound pTyr came from the six-residue libraries and contain two positively charged amino acids at 55_{H} and 56_{H}. The H2 sequences of pSAb, pSTAb, and pYAb are ATGGHT, STPRGST, and VTGGRK, respectively.

FIG. 4 illustrates Biacore traces of phospho-specific Fabs binding to phosphorylated peptides. (a) pSAb binding to pSer peptide. (b) pSTAb binding to the pSer peptide. (c) pSTAb binding to the pThr peptide. (d) pYAb binding to the pTyr peptide.

FIG. 5 illustrates density maps of Fab structures. Strong electron density in pSAb:pSer (a), pSTAb:pSer (b), pSTAb:pThr (c), and pYAb:pTyr (d) complexes was observed for the peptide (top panels Fo-Fc maps) and for the phosphoresidue and CDR H2 loop (H50-H56) (bottom panels 2Fo-Fc maps). Fo-Fc maps were contoured to 3σ and 2Fo-Fc maps were contoured to 1.25σ.

FIG. 6 illustrates the structural comparison between the mouse and humanized Fab (a), and the bound and unbound Fab (b). (a) Alignment of the mouse Fab with the humanized Fab reveals no major deviations in the position of CDRs between the Fabs (ca RMSD of 0.78 Å). (b) Comparison of the unbound and bound forms of pYAb reveals no major shifts in CDR position upon binding to the peptide (cα RMSD of 1.3 Å).

FIG. 7 illustrates the X-ray crystal structures of phosphoresidue-binding hot spot from pSAb (a), pSTAb (b and c), and pYAb (d). (a) In pSAb, pSer makes hydrogen bonds with all three specificity residues (G53, H55 and T56). The anchoring hydrogen bond to T52A is conserved. (b and c) In pSTAb, the pSer/pThr makes hydrogen bonds with two specificity residues (R53 and S55), one anchor residue (S52), and the conformation residue (G54). In both pSTAb structures bound to pSer and pThr, R53 forms a bidentate interaction with the phosphate. The anchor residue T52A is flipped compared to pSAb, which allows the backbone carbonyl to make a new anchoring hydrogen bond. (d) The pTyr is recognized by a salt bridge with K56 and a hydrophobic interaction between V52 and the phenyl ring of the pTyr. However, longer side chain of pTyr prevents the phosphate group from occupying the phosphate-binding pocket, which is instead occupied by a water molecule (shown as a sphere). (e) The structures demonstrate two distinct recognition modules: a phosphoresidue-binding pocket and peptide-binding region.

FIG. 8 illustrates the electrostatic surface representations of the parent Fab (a), pSAb (b), pSTAb (c), and pYAb (d). Comparison of phosphate-binding pocket (indicated by arrow) in CDR H2 reveals larger electropositive pocket for all the phospho-specific scaffolds. Surfaces were calculated with APBS and generated with MacPymol.

FIG. 9 illustrates the generation of novel recombinant phospho-specific (PS) antibodies using the pSAb and pSTAb scaffolds. (a) Representative phage ELISAs of one scFv clone selected against each of the nine phosphopeptide targets demonstrates that PS Abs to seven out of the ten targets were selected. No hits were observed against P7. To analyze target specificity, the binding of each scFv to ten different phosphopeptides was characterized by phage ELISA (b). Representative results are shown for the scFv clones that were selected against P10. (c) Heatmap representation of the phage ELISA binding signals for each scFv (vertical axis) against each of the ten phosphopeptides (horizontal axis). For each scFv, signals were normalized to the highest overall ELISA signal observed against the ten peptides. The scale goes from zero to one. (d) Competition ELISA data for two of the scFv-Fc fusions demonstrates high affinities for the target phosphopeptides. Calculated K_{D} values for every clone analyzed are shown in Table 11.

FIG. 10 illustrates the phosphoresidue-binding pocket from natural phosphopeptide binding domains. In all structures, at least one Lys or Arg makes a salt bridge with the phosphoresidue. The CDR H2 pocket from a scFv isolated from an immunized chicken that binds to a pThr-containing peptide is also shown (73). Representative structures for the 14-3-3, WW, BRCA1 C-terminus (BRCT), WD40, forkhead-associated (FHA), Src Homology 2 (SH2), and phosphotyrosine-binding (PTB) domains are from PDB ID 1ywt (74), 1f8a (75), 1t15 (76), 1nex (77), 1j41 (78), 1a0t (79), and 1shc (80), respectively.

FIG. 11 provides an alignment of the exemplary antibody sequences identified employing the methods described in the present disclosure.

### DETAILED DESCRIPTION OF THE INVENTION

### Introduction

Traditional polyclonal Abs generation using immunization and in vitro Ab generation methods fail to generate high affinity post-translational modification-specific Abs because most native Abs do not possess any initial affinity for the small peptide antigens harboring the post-translational modification. The invention described herein provides high affinity Abs using a novel structure-guided Ab generation strategy that employs Ab scaffolds with engineered or identified hot spots tailored to bind to a sequence motif specific to a particular post-translational modification.

Provided herein are methods and compositions wherein a motif-specific anchoring hot spot that recognizes a specific post-translational protein modification is first engineered or identified in a CDR of a parent antibody, thus creating an antibody scaffold. The sequence-specific designs of the present disclosure are termed “hot spots” or binding pockets or anchoring pockets because they contribute a substantial fraction of the binding energy to a protein-protein interaction (Clackson, T. & Wells, J. A. A hot spot of binding energy in a hormone-receptor interface. Science 267, 383-386 (1995); Bogan, A. A. & Thorn, K. S. Anatomy of hot spots in protein interfaces. J Mol Biol 280, 1-9 (1998)). The antibody scaffold that includes the hot spot provides an initial antigen-binding affinity to the desired modification. Following generation of the Ab scaffold, diverse Ab libraries can then be used for in vitro selection wherein CDR positions are randomized outside of the engineered anchoring hot spot. Selections with these libraries allow a skilled artisan to isolate novel antibodies that are highly specific against peptides harboring the desired post-translational modification.

### DEFINITIONS

Unless specifically indicated otherwise, all technical and scientific terms used herein have the same meaning as commonly understood by those of ordinary skill in the art to which this invention belongs. In addition, methods or materials that are substantially equivalent to a method or material described herein can be used in the practice of the present invention. For purposes of the present invention, the following terms are defined.

The phrase “noncovalent, post translational modification-binding motif” refers to amino acids that adopt a noncovalent conformation that recognizes a post translational modification. Such motifs are present in protein domains that recognize protein modifications such as phosphate, sulfate, acetyl, methyl, nitric oxide, N-terminal alpha amine, C-terminal carboxylate, GalNAc sugar or GlcNAc sugar. These motifs are used to identify or design “hot spots” or “anchoring pocket sequences” that are incorporated into antibody CDRs and used to specifically bind post translationally modified proteins.

The term “anchoring pocket sequence” or “anchoring pocket” or “hot spot” refers to a non-covalent conformation of a subsequence or portion of one or more antibody CDRs that is specific to and binds to a protein post translational modification. The anchoring pocket sequence can be present in a known CDR or can be engineered. The anchoring pocket sequence is optionally further modified to have more specific binding properties for the protein post translational modification. Essentially, the noncovalent, post translational modification-binding motif (found in a protein domain) and the anchoring pocket sequence (found in a CDR or CDRs) perform the same function of binding to post translational modifications.

As used herein, an antibody scaffold is an antibody or antibody fragment that includes a CDR or more than one CDR that forms an anchoring pocket sequence, or “hot spot.”

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized. Antibodies exist, e.g., as intact immunoglobulins or as a number of well-characterized fragments produced by digestion with various peptidases (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv or Fab fragments) or those identified using phage display libraries (see, e.g., McCafferty et al., Nature 348:552-554 (1990)).

The term “CDR” as used herein specifies the Complementarity Determining Region (see, for example, Harlow and Lane, “Antibodies, a Laboratory Manual,” CSH Press, Cold Spring Harbour, 1988). A CDR is a relatively short amino acid sequence found in the variable (V) domains of an antibody. Each variable domain (the heavy chain VH and light chain VL) of an antibody comprises three complementarity determining regions sometimes called hypervariable regions, flanked by four relatively conserved framework regions or “FRs.” The six CDRs of an antibody essentially determine the specificity of an antibody and make the contact with a specific ligand.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

In some embodiments, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

In some embodiments, the antibody can be a mixture from different species, e.g. a chimeric antibody and/or a humanized antibody. That is, in the present invention, the CDR sets can be used with framework and constant regions other than those specifically described by sequence herein.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. application Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. application Ser. No. 09/810,510; Tan et al., 2002, J. Immunol. 169:1119-1125; De Pascalis et al., 2002, J. Immunol. 169:3076-3084, all entirely incorporated by reference.

In some embodiments the antibodies are diabodies. In some embodiments, the antibody is a minibody. Minibodies are minimized antibody-like proteins comprising a scFv joined to a CH3 domain. Hu et al., 1996, Cancer Res. 56:3055-3061, entirely incorporated by reference. In some embodiments, the scFv can be joined to the Fc region, and may include some or the entire hinge region.

The term “antigen” refers to a molecule capable of being bound by an antibody. An antigen is additionally capable of inducing a humoral immune response and/or cellular immune response leading to the production of B and/or T-lymphocytes.

The term “diversifying” or “to diversify” or “diversification” as used herein refers to a method of enhanced sequence evolution to generate a library of variants having unique amino acid sequence signatures. Protein diversification or evolution is well known in the art.

The term “variant” as used here refers to a polypeptide, protein, amino acid sequence, Fab, or antibody that is modified from its parental precursor.

The term “polypeptide” or “protein” refers to a polymer of two or more amino acid residues. The terms apply to amino acid polymers in which one or more amino acid residue is an artificial, chemical analogue of a corresponding naturally occurring amino acid, as well as to polymers of naturally occurring amino acids. The term “recombinant protein” refers to a protein that is produced by expression of a nucleotide sequence encoding the amino acid sequence of the protein from a recombinant DNA molecule.

The term “target peptide” as used herein refers to a peptide comprising an amino acid sequence recognized by the engineered antibodies described herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such unnatural amino acid analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid substitution” as used herein refers to the deletion and/or replacement of a specific acid of a given position.

The term “post-translationally modified amino acid” as used herein refers to an amino acid that has been modified following ribosomal translation. Non-limiting examples of post-translational modifications are phosphorylated amino acids, e.g., phosphotyrosine (pTry), phosphoserine (pSer), and phosphothreonine (pThr). Other post-translational modifications can include oxidized cysteine, sulfated tyrosine, or acetylated lysine.

The term “naturally occurring” is used to refer to a protein, nucleic acid molecule, cell, or other material that exists in the natural world, for example, a polypeptide or polynucleotide sequence that is present in an organism, including in a virus. In general, at least one instance of a naturally occurring material existed in the world prior to its creation, duplication, or identification by a human. A naturally occurring material can be in its form as it exists in the natural world, or can be modified by the hand of man such that, for example, it is in an isolated form.

The term “nucleic acid” refers to deoxyribonucleotides or ribonucleotides and polymers thereof in either single- or double-stranded form, and complements thereof. The term refers to all forms of nucleic acids (e.g., gene, pre-mRNA, mRNA) and their polymorphic variants, alleles, mutants, and interspecies homologs. The term nucleic acid is used interchangeably with gene, cDNA, mRNA, oligonucleotide, and polynucleotide. The term encompasses nucleic acids that are naturally occurring or recombinant. Nucleic acids can (1) code for an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. Nucleic acid symbols used herein are the standard IUPAC naming for nucleic acids, as exemplified in the following Table 1:

TABLE 1
IUPAC Nucleic Acid Naming

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | gap |

The term “recombinant nucleic acid molecule” refers to a non-naturally occurring nucleic acid molecule containing two or more linked polynucleotide sequences. A recombinant nucleic acid molecule can be produced by recombination methods, particularly genetic engineering techniques, or can be produced by a chemical synthesis method. A recombinant nucleic acid molecule can encode a fusion protein, for example, a fluorescent protein variant of the invention linked to a polypeptide of interest. The term “recombinant host cell” refers to a cell that contains a recombinant nucleic acid molecule. As such, a recombinant host cell can express a polypeptide from a “gene” that is not found within the native (non-recombinant) form of the cell.

Reference to a polynucleotide “encoding” a polypeptide means that, upon transcription of the polynucleotide and translation of the mRNA produced there from, a polypeptide is produced. The encoding polynucleotide is considered to include both the coding strand, whose nucleotide sequence is identical to an mRNA, as well as its complementary strand. It will be recognized that such an encoding polynucleotide is considered to include degenerate nucleotide sequences, which encode the same amino acid residues. Nucleotide sequences encoding a polypeptide can include polynucleotides containing introns as well as the encoding exons.

An expression control sequence refers to a nucleotide sequence that regulates the transcription or translation of a polynucleotide or the localization of a polypeptide to which it is operatively linked expression control sequences are “operatively linked” when the expression control sequence controls or regulates the transcription and, as appropriate, translation of the nucleotide sequence (i.e., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a cell compartmentalization signal, which can target a polypeptide to the cytosol, nucleus, plasma membrane, endoplasmic reticulum, mitochondrial membrane or matrix, chloroplast membrane or lumen, medial trans-Golgi cistemae, or a lysosome or endosome. Cell compartmentalization domains are well known in the art and include, for example, a peptide containing amino acid residues 1 to 81 of human type II membrane-anchored protein galactosyltransferase, or amino acid residues 1 to 12 of the presequence of subunit IV of cytochrome c oxidase (see also Hancock et al., EMBO J. 10:4033-4039, 1991; Buss et al., Mol. Cell. Biol. 8:3960-3963, 1988; and U.S. Pat. No. 5,776,689; each of which is incorporated herein by reference).

The term “immunoassay” refers to an assay that utilizes an antibody to specifically bind an analyte. An immunoassay is characterized by the use of specific binding properties of a particular antibody to isolate, to target, or to quantify the analyte.

The term “identical” or “identity” or “percent identity,” or “sequence identity” in the context of two or more nucleic acids or polypeptide sequences that correspond to each other refer to two or more sequences or subsequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” and are embraced by the term “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists for a specified entire sequence or a specified portion thereof or over a region of the sequence that is at least about 25 amino acids or nucleotides in length, or more preferably over a region that is 50-100 amino acids or nucleotides in length. A corresponding region is any region within the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. in some embodiments, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

In some embodiments, an exemplary algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “conservatively modified variation,” when used in reference to a particular polynucleotide sequence, refers to different polynucleotide sequences that encode identical or essentially identical amino acid sequences, or where the polynucleotide does not encode an amino acid sequence, to essentially identical sequences. Because of the degeneracy of the genetic code, a large number of functionally identical polynucleotides encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleotide sequence variations are “silent variations,” which can be considered a species of “conservatively modified variations.” As such, it will be recognized that each polynucleotide sequence disclosed herein as encoding a fluorescent protein variant also describes every possible silent variation. It will also be recognized that each codon in a polynucleotide, except AUG, which is ordinarily the only codon for methionine, and UUG, which is ordinarily the only codon for tryptophan, can be modified to yield a functionally identical molecule by standard techniques. Accordingly, each silent variation of a polynucleotide that does not change the sequence of the encoded polypeptide is implicitly described herein.

Furthermore, it will be recognized that individual substitutions, deletions or additions that alter, add or delete a single amino acid or a small percentage of amino acids (typically less than 5%, and generally less than 1%) in an encoded sequence can be considered conservatively modified variations, provided alteration results in the substitution of an amino acid with a chemically similar amino acid. Conservative amino acid substitutions providing functionally similar amino acids are well known in the art, including the following six groups, each of which contains amino acids that are considered conservative substitutes for each another:

1) Alanine (Ala, A), Serine (Ser, S), Threonine (Thr, T);

2) Aspartic acid (Asp, D), Glutamic acid (Glu, E);

3) Asparagine (Asn, N), Glutamine (Gln, Q);

4) Arginine (Arg, R), Lysine (Lys, K)

5) Isoleucine (Ile, I), Leucine (Leu, L), Methionine (Met, M), Valine (Val, V); and

6) Phenylalanine (Phe, F), Tyrosine (Tyr, Y), Tryptophan (Trp, W).

Two or more amino acid sequences or two or more nucleotide sequences are considered to be “substantially identical” or “substantially similar” if the amino acid sequences or the nucleotide sequences share at least 80% or 90% sequence identity with each other, or with a reference sequence over a given comparison window. Thus, substantially similar sequences include those having, for example, at least 80% sequence identity, at least 90% sequence identity, at least 95% sequence identity, at least 97% sequence identity, at least 98% sequence identity, or at least 99% sequence identity.

The term “substitution” refers to includes the replacement of one or more amino acid residues either by other naturally occurring amino acids, (conservative and non-conservative substitutions), by non-naturally occurring amino acids (conservative and non-conservative substitutions), or with organic moieties which serve either as true peptidonimetics (e.g., having the same steric and electrochemical properties as the replaced amino acid), or merely serve as spacers in lieu of an amino acid, so as to keep the spatial relations between the amino acid spanning this replaced amino acid.

### Antibody Scaffolds

An antibody scaffold is a region of an antibody or antibody fragment comprising one or more CDRs that have anchoring pocket sequences or hot spots that provide an initial binding affinity to a specific post-translational modification. For example, an antibody scaffold can include a hot spot that is specific to a phosphorylated amino acid. The host spot or anchoring pocket can be further engineered or tailored to provide additional affinity for the post translational modification. The antibody scaffolds thus provide an initial template to allow in vitro selection and diversification, as described in detail below, for the generation of novel antibodies specific to the post-translational modification.

One of skill in the art would recognize that the antibody scaffolds described herein can be engineered for specificity towards any post-translational modifications that can include, but is not limited to, myristoylation, palmitoylation, isoprenylation, prenylation, farnesylation, geranylgeranylation, glypiation, glycosylphosphatidylinositol anchor formation, lipoylation, flavin attachment, heme C attachment, phosphopantetheinylation, retinylidene Schiff base formation, diphthamide formation, ehtanolamine phosphoglycerol attachment, hypusin formation, acylation, O-acylation, N-acylation, sacylation, acetylation, histone acetylation, deacetylation, formylation, aklylation, methylation, amide bond formation, amidation, arginylation, polyglutamylation, polyglycylation, butyrylation, gamma-carboxylation, glycosylation, polysialylation, malonylation, hydroxylation, iodination, ribosylation, oxidation, phosphate ester or phosphoramidate formation, phosphorylation, adenylation, propionylation, pyroglutamate formation, S-glutathionylation, S-nitrosylation, succinylation, sulfation, selenoylation, glycation, biotinylation, pegylation, ISGylation, SUMOylation, ubiquitination, neddylation, pupylation, citrullination, deamidation, eliminylation, carbamylation, disulfide bridge formation, proteolytic cleavage, or racemization.

Antibody scaffolds are engineered by identifying a suitable parent antibody scaffold that contains or can be used to install the sequence motif hot spot using standard recombinant technology techniques as described in detailed below. The scaffold itself can also be mutated by any method well known in the art as described herein to increase the specificity to its particularly designed hot spot prior to in vitro selection.

The antibody scaffolds described herein can use any antibody known in the art. The antibody scaffolds can further be used to create antibody fragments, such as bispecific antibodies, fragment antigen binding (Fab), trifunctional antibodies, single-chain variable fragments, single domain antibody, bi-specific T-cell engagers, and the like. Humanized or primatized antibodies can be used to create the antibody scaffolds described herein. Generally, a humanized antibody has one or more amino acid residues introduced into it from a source which is non-human. These non-human amino acid residues are often referred to as import residues, which are typically taken from an import variable domain. Methods for humanizing or primatizing non-human antibodies are well known in the art. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. These references are hereby incorporated by reference in their entirety for all purposes and in particular for all teaching related to constructing antibody scaffolds. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

### Construction of Antibody Scaffold Vectors

The antibody scaffold of the present invention can be generated using vector construction and expression protocols well known in the art. For example, to obtain high level expression of a cloned gene or genome, one typically subclones the nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described (e.g., in Sambrook et al., and Ausubel et al., supra. Bacterial expression systems for expressing the protein are available in, e.g., E. coli, Bacillus sp., and Salmonella (Palva et al., Gene 22:229-235 (1983)); Mosbach et al., Nature 302:543-545 (1983)), which are hereby incorporated by reference in their entirety for all purposes and in particular for all teaching related to constructing antibody scaffold vectors. Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Heterologous refers to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). Additionally, phoA promoters can be used for phage display vectors described below.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. Additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

The particular expression vector used to transport the genetic information into the cell is not particularly critical. Any of the conventional vectors used for expression in eukaryotic or prokaryotic cells can be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags can be included in an expression cassette for nucleic acid rescue. Markers such as fluorescent proteins, green or red fluorescent protein, 13-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMT010/A⁺, pMAMneo-5, baculovirus pDSVE, pJK1, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline, by the incorporation of response elements for these agents into the promoter. Generally, high level expression is obtained from inducible promoters only in the presence of the inducing agent; basal expression levels are minimal.

Vectors can have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, as any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)), which are hereby incorporated by reference in their entirety for all purposes and in particular for all teaching related to constructing antibody scaffold vectors. Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (1983)).

Any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the antibody scaffold proteins of the present invention.

The vectors of the present invention can be subjected to recombination and/or mutagenesis. This can be achieved by a variety of methods known in the art. One such non-limiting method is SOE-PCR as described by Kirchhoff and Desrosiers, PCR Methods and Applications 2:301-304 (1993). Another method is Kunkel mutagenesis, which is a type of site-directed mutagenesis that employs multiple mutagenic primers to generate libraries with multiple mutated positions. This is achieved by (a) generating uracil containing nucleotide template encoding a polypeptide of interest (b) synthesizing 2-50 mutagenic primers corresponding to at least one region of identity in the nucleotide template, wherein each mutagenic primer comprises at least one substitution of the template sequence (or: insertion/deletion of bases) resulting in at least one amino acid substitution (or insertion/deletion) in the amino acid sequence encoded by the starting primer, (c) contacting the mutagenic primers with the template of (a) under conditions wherein a mutagenic primer anneals to the template sequence and extension of the primers are catalyzed by a polymerase to generate a mixture of mutagenized polynucleotides and uracil-containing templates, and (d) transforming a host cell with the polynucleotide and template mixture wherein the template is degraded and the mutagenized polynucleotide replicated and thus generating a library of polynucleotide variants of the gene of interest. DNA shuffling can also be used to prepare a mutated library. DNA shuffling is the (partially) random process in which a library of chimeric genes is generated from two or more starting genes. The starting genes are heterologous such that one gene is different from any other starting gene in at least one nucleotide; e.g., the starting material can be point mutations of each other. Much more diversity can be included in the process if the parental genes differ in more positions, e.g. by representing genes encoding homologues of proteins having the same function (and structural family) but originating from different species. The latter experiment has been denoted “family shuffling” (Crameri et al., Nature, 391: 288-291 (1998)). A number of other formats of carrying out, shuffling, recombination, or site-directed mutagenesis process have been described.

After the expression vector is introduced into the cells, the transfected cells are cultured under conditions favoring expression of the protein of choice, which is recovered from the culture using standard techniques identified below.

Either naturally occurring or recombinant proteins can be purified for use in diagnostic assays, for making antibodies (for diagnosis and therapy) and vaccines, and for assaying for anti-viral compounds. Naturally occurring protein can be purified, e.g., from primate tissue samples. Recombinant protein can be purified from any suitable expression system.

### Purification of Antibody Scaffold Proteins

The antibody scaffold proteins can be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra), which are hereby incorporated by reference in their entirety for all purposes and in particular for all teaching related to constructing antibody scaffold vectors.

A number of procedures can be employed when recombinant protein is being purified. For example, proteins having established molecular adhesion properties can be reversibly fused to the protein. With the appropriate ligand or substrate, a specific protein can be selectively adsorbed to a purification column and then freed from the column in a relatively pure form. The fused protein is then removed by enzymatic activity. Finally, protein can be purified using immunoaffinity columns. Recombinant protein can be purified from any suitable source, include yeast, insect, bacterial, and mammalian cells.

Recombinant proteins can be expressed and purified by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria can form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra). If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility fractionation can be used as a standard protein separation technique for purifying proteins. As an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The protein can also be separated from other proteins on the basis of its size, net surface charge, hydrophobicity, and affinity for ligands or substrates using column chromatography. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### In Vitro Section for Antibody Diversification

In an embodiment, following the generation of an antibody scaffold having an engineered anchoring hot spot specific to a particular sequence motif, regions outside of the engineered hot spot can be mutated and selected for binding to other motif-containing targets. This aspect of the invention includes evolving the scaffold through screening and selection of antibody libraries.

Methods for high throughput screening of antibody libraries can include, but are not limited to, display techniques including cell display, bacterial display, yeast display, mammalian display, ribosome display, mRNA display, and phage display. The use of phage display to isolate ligands that bind biologically relevant molecules has been reviewed in Felici et al. (1995) Biotechnol. Annual Rev. 1:149-183, Katz (1997) Annual Rev. Biophys. Biomol. Struct. 26: 27-45 and Hoogenboom et al. Immunotechnology 4(1): 1-20 (1998). Several randomized combinatorial peptide libraries have been constructed to select for polypeptides that bind different targets, e.g. cell surface receptors or DNA (reviewed by Kay, Perspect. Drug Discovery Des. 2, 251-268 (1995); Kay and Paul, Mol. Divers. 1: 139-140 (1996)). Proteins and multimeric proteins have been successfully phage-displayed as functional molecules (see EP 0349578A, EP 4527839A, EP 0589877A; Chiswell and McCafferty, Trends Biotechnol. 10, 80-84 (1992)). In addition, functional antibody fragments (e.g. Fab, single chain Fv [scFv]) have been expressed (McCafferty et al., Nature 348: 552-554 (1990); Barbas et al., Proc. Natl. Acad Sci. USA 88: 7978-7982 (1991); Clackson et al., Nature 352: 624-628 (1991)). These references are hereby incorporated by reference in their entirety for all purposes and in particular for all teaching related to antibody diversification as described herein.

An exemplary method for in vitro protein evolution of the Ab scaffolds of the present invention is phage display, and phage display methods are well known in the art. Phage display libraries can be created by making a designed series of mutations or variations within a coding sequence for the Ab scaffold template, each mutant sequence encoding an amino acid corresponding in overall structure to the template except having one or more amino acid variations in the sequence of the template. Retroviral and phage display vectors can be engineered using standard vector construction techniques well known in the art, as described herein relating to antibody scaffold construction. P3 phage display vectors along with compatible protein expression vectors, as is well known in the art, can be used to generate phage display vectors for antibody diversification as described herein.

The novel variegated (mutated) DNA provides sequence diversity, and each transformant phage displays one variant of the initial template amino acid sequence encoded by the DNA, leading to a phage population (library) displaying a vast number of different but structurally related amino acid sequences. The amino acid variations are expected to alter the binding properties of the binding peptide or domain without significantly altering its structure, at least for most substitutions.

In a typical screen, a phage library is contacted with and allowed to bind the target, or a particular subcomponent thereof. To facilitate separation of binders and non-binders, it is convenient to immobilize the target on a solid support. Phage bearing a target-binding moiety can form a complex with the target on the solid support whereas non-binding phage remain in solution and can be washed away with excess buffer. Bound phage are then liberated from the target by changing the buffer to an extreme pH (pH 2 or pH 10), changing the ionic strength of the buffer, adding denaturants, or other known means. To isolate the binding phage exhibiting the polypeptides of the present invention, a protein elution is performed.

The recovered phage can then be amplified through infection of bacterial cells and the screening process can be repeated with the new pool that is now depleted in non-binders and enriched for binders. The recovery of even a few binding phage is sufficient to carry the process to completion. After a few rounds of selection, the gene sequences encoding the binding moieties derived from selected phage clones in the binding pool are determined by conventional methods, described below, revealing the peptide sequence that imparts binding affinity of the phage to the target. When the selection process works, the sequence diversity of the population falls with each round of selection until desirable binders remain. The sequences converge on a small number of related binders, typically 10-50 out of about 10⁹ to 10¹⁰ original candidates from each library. An increase in the number of phage recovered at each round of selection is a good indication that convergence of the library has occurred in a screen. After a set of binding polypeptides is identified, the sequence information can be used to design other secondary phage libraries, biased for members having additional desired properties.

### Characterizing Antibody Scaffolds

The antibody scaffolds of the present invention can be characterized for binding to modified antigens. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. (See, e.g., Self et al., Curr. Opin. Biotechnol 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); immunohistochemical assay, capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. (See, e.g., Schmalzing et al., Electrophoresis, 25 18:2184-93 (1997); Bao, J Chromatogr. B. Biomed. Sci., 699:463-80 (1997)). Liposome immunoassays, such as flow-injection liposome immunoassays and liposome immunosensors, are also suitable for use in the present invention. (See, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J Clin. Chem. Clin. Biochem., 27:261-276 (1989)). These references are hereby incorporated by reference in their entirety for all purposes and in particular for all teaching related to characterizing the antibody scaffolds described herein.

Specific immunological binding of an antibody can be detected directly or indirectly. A detectable moiety can be used in the assays described herein (direct or indirect detection). A variety of detectable moieties are well known to those skilled in the art, and can be any material detectable by spectroscopic, photochemical, biochemical, immunochemical, electrical, optical or chemical means. Detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, metals, and the like. Direct labels include fluorescent or luminescent tags, metals, dyes, radionucleodies, and the like, attached to the antibody. An antibody labeled with iodine-125 (1251) can be used. A chemiluminescence assay using a chemiluminescent antibody specific for nucleic acids or proteins is suitable for sensitive, non-radioactive detection of nucleic acids or protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urebromocresol purple (Sigma Immunochemicals; St. Louis, Mo.). Other proteins capable of specifically binding immunoglobulin constant regions, such as protein A or protein G can also be used as a label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985), which are hereby incorporated by reference in their entirety for all purposes and in particular for all teaching related to characterizing the antibody scaffolds described herein.

fluorophores are known in the art and can be used in the present invention.

In some embodiments, a number of fluorescent molecules can be employed with the methods of the present disclosure. In some embodiments, the fluorophore exhibits green fluorescence (such as for example 494 nm/519 nm), orange fluorescence (such as for example 554 nm/570 nm), red fluorescence (such as for example 590 nm/617 nm), or far red fluorescence (such as for example 651 nm/672 nm) excitation/emission spectra. In some embodiments, the fluorophore is a fluorophore with excitation and emission spectra in the range of about 350 nm to about 775 nm. In some embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm or about 749 nm/775 nm. In some embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In some embodiments, the fluorophore can include but is not limited to Cy dyes, including Cy2, Cy3, Cy3B, Cy3.5, Cy5, Cy5.5 and Cy7 (available from GE Life Sciences or Lumiprobes). In some embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In some embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); 6-FAM (6-Carboxyfluorescein), 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR1 1O (5-Carboxyrhodamine 110); 6-CR1 1O (6-Carboxyrhodamine 110); 5-CR6G (5-Carboxyrhodamine 6G); 6-CR6G (6-Carboxyrhodamine 6G); 5(6)-Caroxyrhodamine 6G cadaverine; 5(6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 malemide; 6-TMR C6 malemide; TR C2 malemide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

Western blot (immunoblot) analysis can be used to detect and quantify the presence of an antigen in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the antigen. The anti-antigen antibodies specifically bind to the antigen on the solid support. These antibodies can be directly labeled or alternatively can be subsequently detected using labeled antibodies (e.g., labeled sheep anti-mouse antibodies) that specifically bind to the anti-antigen antibodies.

An ELISA method can be used as follows: (1) bind an antibody or antigen to a substrate; (2) contact the bound receptor with a fluid or tissue sample containing the virus, a viral antigen, or antibodies to the virus; (3) contact the above with an antibody bound to a detectable moiety (e.g., horseradish peroxidase enzyme or alkaline phosphatase enzyme); (4) contact the above with the substrate for the enzyme; (5) contact the above with a color reagent; (6) observe color change.

An antigen and/or a patient's antibodies to the virus can be detected utilizing a capture assay. Briefly, to detect antibodies in a sample, antibodies to an immunoglobulin, e.g., anti-IgG (or IgM) are bound to a solid phase substrate and used to capture the patient's immunoglobulin from serum. The antigen, or reactive fragments of the antigen, is then contacted with the solid phase followed by addition of a labeled antibody. The amount of specific antibody can then be quantitated by the amount of labeled antibody binding. A micro-agglutination test can also be used to detect the presence of an antigen in test samples. Briefly, latex beads are coated with an antibody and mixed with a test sample, such that the antigen in the tissue or body fluids that is specifically reactive with the antibody crosslinked with the receptor, causing agglutination. The agglutinated antibody-virus complexes within a precipitate, visible with the naked eye or by spectrophotometer.

Competitive assays can also be adapted to provide for an indirect measurement of the amount of an antigen present in the sample. Briefly, serum or other body fluids from the subject is reacted with an antibody bound to a substrate (e.g. an ELISA 96-well plate). Excess serum is thoroughly washed away. A labeled (enzyme-linked, fluorescent, radioactive, etc.) monoclonal antibody is then reacted with the previously reacted antibody complex. The amount of inhibition of monoclonal antibody binding is measured relative to a control. Monoclonal antibodies (MABs) can also be used for detection directly in samples by IFA for MABs specifically reactive for the antibody-antigen complex.

A hapten inhibition assay is another competitive assay. In this assay the known antigen can be immobilized on a solid substrate. A known amount of anti-antigen antibody is added to the sample, and the sample is then contacted with the immobilized antigen. The amount of antibody bound to the known immobilized antigen is inversely proportional to the amount of antigen present in the sample. The amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

Immunoassays in the competitive binding format can also be used for crossreactivity determinations. For example, an antigen can be immobilized to a solid support. Proteins can be added to the assay that compete for binding of the antisera to the immobilized antigen. The ability of the added proteins to compete for binding of the antisera to the immobilized protein is compared to the ability of the antigen to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs. The immunoabsorbed and pooled antisera can then be used in a competitive binding immunoassay as described above to compare a second protein, thought to be perhaps an allele or polymorphic variant of an antigen, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the antigen that is required to inhibit 50% of binding, then the second protein is said to specifically bind to the polyclonal antibodies generated to antigen.

A signal from a direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. Where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Colorimetric or chemiluminescent labels can be detected simply by observing the color associated with the label. Thus, in various dipstick assays, conjugated gold often appears pink, while various conjugated beads appear the color of the bead. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measureable signal, such as a colored spot.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

In some embodiments, immunoprecipitation can be used to detect and quantify the presence of antibody-antigen binding. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria. The antibodies can also be coupled to sepharose beads that can easily be isolated out of solution. After washing, the precipitate can be analyzed using mass spectrometry, Western blotting, or any number of other methods for identifying constituents in the complex.

### Structural Analysis of Antibody Scaffolds

Recognition of post-translation modification by the Ab scaffolds of the present invention can be explored by any structural analysis tools known in the art, e.g., X-ray crystallography. X-ray crystallography requires that an Ab scaffold be expressed and purified as described herein, and crystallized. Crystallization conditions and cryoprotectant solutions are listed in Table 7 below. Generally, a subject Ab scaffold can be crystallized using any of a variety of crystallization methods known in the art, many of which are reviewed in Caffrey (J. Struct. Biol. 142:108-32 (2003)). Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege and Ducruix (1999) Chapter 1, In Crystallization of Nucleic Acids and Proteins, a Practical Approach, 2nd ed., (Ducruix and Giege, eds.) (Oxford University Press, New York, 1999) pp. 1-16. Crystallization of antibodies and antibody fragments is well known in the art, see e.g. Shenoy et al. (WO/2002/072636). These references are hereby incorporated by reference in their entirety for all purposes and in particular for all teaching related to characterizing the antibody scaffolds described herein. In addition, protein structures can be determined by neutron diffraction and nuclear magnetic resonance.

### Pharmaceutical Compositions

Pharmaceutical compositions within the scope of the present invention can also contain other compounds, which can be biologically active or inactive. For example, one or more immunogenic portions of other antigens can be present, either incorporated into a fusion polypeptide or as a separate compound, within the composition or vaccine. Polypeptides can, but need not be, conjugated to other macromolecules as described, for example, within U.S. Pat. Nos. 4,372,945 and 4,474,757. Pharmaceutical compositions and vaccines can generally be used for prophylactic and therapeutic purposes.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for parenteral administration, for example, by intraarticular (in the joints), intravenous, intramuscular, intradermal, intraperitoneal, and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to affect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17th ed., 1989). Administration can be in any convenient manner, e.g., by injection, oral administration, inhalation, transdermal application, or rectal administration.

### Methods of Identifying Antibodies

In some embodiments, the present invention provides a method for identifying antibodies that specifically bind a protein comprising a post translational modification, the method comprising the steps of: (1) identifying a noncovalent post translational modification-binding motif in a protein; (2) inspecting known antibody CDRs for an anchoring pocket sequence that adopts the conformation of the noncovalent post translational modification-binding motif; (3) preparing an antibody scaffold comprising a CDR, wherein amino acid residues from the CDR comprise the anchoring pocket; (4) preparing a library comprising the antibody scaffold, wherein the CDR is randomized outside of the anchoring pocket; and (5) identifying antibodies in the library that specifically bind to a protein comprising a post translational modification.

In some embodiments, identifying a noncovalent post translational modification-binding motif in a protein can be performed using any of the methods described herein as well as any other methods known to those of skill in the art.

In some embodiments, the methods further comprise analysis which is performed according the present methods by inspecting known antibody CDRs for an anchoring pocket sequence that adopts the conformation of the noncovalent post translational modification-binding motif. CDRs can be screened by any of the methods described herein, including in vitro and in silico methods, as well as any other methods known to those of skill in the art for antibody screening in order to identify anchoring pocket sequences that adopt the conformation of the noncovalent post translational modification-binding motif.

In some embodiments, the methods further comprise preparing an antibody scaffold comprising a CDR, wherein amino acid residues from the CDR comprise the anchoring pocket identified according to the present methods. In some embodiments, the antibody scaffold comprises two or more CDRs and amino acid residues from the two or more CDRs comprise the anchoring pocket. In some embodiments, the antibody scaffold comprises two, three, four or more CDRs and amino acid residues from the two, three, four or more CDRs comprise the anchoring pocket. In some embodiments, the antibody scaffold comprises two CDRs and amino acid residues from the two CDRs comprise the anchoring pocket. In some embodiments, the antibody scaffold comprises three CDRs and amino acid residues from the three CDRs comprise the anchoring pocket. In some embodiments, the antibody scaffold comprises four CDRs and amino acid residues from the four CDRs comprise the anchoring pocket.

In some embodiments, the methods further comprise preparing a library comprising the antibody scaffold, wherein the CDR is randomized outside of the anchoring pocket. In some embodiments, the amino acid residues of the two or more CDRs with amino acids comprising the anchoring pocket are randomized outside of the anchoring pocket. In some embodiments, the amino acid residues of the two, three, four or more CDRs with amino acids comprising the anchoring pocket are randomized outside of the anchoring pocket. In some embodiments, the amino acid residues of the two CDRs with amino acids comprising the anchoring pocket are randomized outside of the anchoring pocket. In some embodiments, the amino acid residues of the three CDRs with amino acids comprising the anchoring pocket are randomized outside of the anchoring pocket. In some embodiments, the amino acid residues of the four CDRs with amino acids comprising the anchoring pocket are randomized outside of the anchoring pocket.

In some embodiments, the methods further comprise identifying antibodies in the library that specifically bind to a protein comprising a post translational modification. In some embodiments, the antibody identified binds a specific protein comprising the post translational modification. In some embodiments, the antibody identified binds a specific post translational modification. In some embodiments, the antibody identified binds a specific post translational modification found in one or more proteins. In some embodiments, the antibody identified binds a specific post translational modification found in a plurality of proteins.

In some embodiments, the antibody identified the post translational modification is any post translation modification know by one of skill in the art. In some embodiments, the post translational modification is an anion or an anionic modification. Examples of post translational modification include but are not limited to phosphorylation, sulfation, acetylation, S-nitrosylation, methylation, proteolysis, or glycosylation. In some embodiments, the post translational modification is selected from the group consisting of phosphorylation, sulfation, acetylation, S-nitrosylation, methylation, proteolysis, and glycosylation. In some embodiments, the post translational modification is a phosphorylation, sulfation, acetylation, S-nitrosylation, methylation, proteolysis, or glycosylation. In some embodiments, post translational modifications can be found in a plurality of proteins.

In some embodiments, the noncovalent post translational modification-binding motif recognizes any post translation modification known by one of skill in the art. In some embodiments, the noncovalent post translational modification-binding motif recognizes an anion or an anionic modification. Examples of noncovalent post translational modification-binding motifs which can be recognized by the antibodies identified by the methods disclosed herein include a phosphate, a sulfate, an acetyl, a methyl, a nitric oxide, a N-terminal alpha amine, a C-terminal carboxylate, a GalNAc sugar or a GlcNAc sugar. In some embodiments, the noncovalent post translational modification-binding motif recognized is a phosphate, a sulfate, an acetyl, a methyl, a nitric oxide, a N-terminal alpha amine, a C-terminal carboxylate, a GalNAc sugar or a GlcNAc sugar.

In some embodiments, the method further comprises the step of characterizing the anchoring pocket. In some embodiments, the amino acid residues from two or more CDRs comprise the anchoring pocket. In some embodiments, the amino acid residues from two, three, four or more CDRs comprise the anchoring pocket. In some embodiments, the amino acid residues from two CDRs comprise the anchoring pocket. In some embodiments, the amino acid residues from three CDRs comprise the anchoring pocket. In some embodiments, the amino acid residues from four CDRs comprise the anchoring pocket.

In some embodiments, the present disclosure provides methods for identifying antibodies that specifically bind a protein comprising a post translational modification, the method comprising the steps of: (1) identifying a noncovalent post translational modification-binding motif in a protein; (2) engineering an antibody scaffold comprising a CDR, wherein amino acid residues from the CDR comprise an anchoring pocket that adopts the conformation of the noncovalent post translational modification-binding motif; (3) preparing a library comprising the antibody scaffold, wherein the CDR is randomized outside of the anchoring pocket; and (4) identifying antibodies in the library that specifically bind to a protein comprising a post translational modification.

In some embodiments, the antibody identified binds a specific protein comprising the post translational modification. In some embodiments, the antibody identified binds a specific post translational modification. In some embodiments, the antibody identified binds a specific post translational modification. In some embodiments, the antibody identified binds a specific post translational modification found in one or more proteins. In some embodiments, the antibody identified binds a specific post translational modification found in a plurality of proteins.

In some embodiments, the antibody identified that specifically bind a protein with the post translational modification binds any post translation modification known by one of skill in the art. In some embodiments, the post translational modification is an anion or an anionic modification. Examples of post translational modification include but are not limited to phosphorylation, sulfation, acetylation, S-nitrosylation, methylation, proteolysis, or glycosylation. In some embodiments, the post translational modification is selected from the group consisting of phosphorylation, sulfation, acetylation, S-nitrosylation, methylation, proteolysis, and glycosylation. In some embodiments, the post translational modification is a phosphorylation, sulfation, acetylation, S-nitrosylation, methylation, proteolysis, or glycosylation.

In some embodiments, the noncovalent post translational modification-binding motif recognizes an anion or an anionic modification. Examples of noncovalent post translational modification-binding motifs which can be recognized by the antibodies identified by the present disclosure include anions, a phosphate, a sulfate, an acetyl, a methyl, a nitric oxide, a N-terminal alpha amine, a C-terminal carboxylate, a GalNAc sugar or a GlcNAc sugar as well as combinations thereof.

In some embodiments, the method further comprises the step of characterizing the anchoring pocket. In some embodiments, the amino acid residues from two or more CDRs comprise the anchoring pocket. In some embodiments, the amino acid residues from two, three, four or more CDRs comprise the anchoring pocket. In some embodiments, the amino acid residues from two CDRs comprise the anchoring pocket. In some embodiments, the amino acid residues from three CDRs comprise the anchoring pocket. In some embodiments, the amino acid residues from four CDRs comprise the anchoring pocket.

In some embodiments, the method of further comprises the step of characterizing the anchoring pocket. The anchoring pocket can be characterized based on structural analysis (including for example but not limited to sequence analysis as well as 3D structure analysis), and functional analysis (including for example binding partner analysis).

In some embodiments, the anchoring pocket can be characterized based on a structural analysis. In some embodiment, the anchoring pocket is characterized based on sequencing analysis. In some embodiments, the anchoring pocket is characterized based on the CDR sequence analysis. In some embodiments, the anchoring pocket is sequenced. In some embodiments, the one, two, three, four or more CDRs in the anchoring pocket are sequenced. In some embodiments, the one CDR in the anchoring pocket is sequenced. In some embodiments, the two CDRs in the anchoring pocket are sequenced. In some embodiments, the three CDRs in the anchoring pocket are sequenced. In some embodiments, the four CDRs in the anchoring pocket are sequenced.

In some embodiments, the anchoring pocket is characterized based on a functional analysis. In some embodiments, the anchoring pocket is characterized based on the noncovalent post translational modification-binding motif recognized by the anchoring pocket. In some embodiments, characterized of the anchoring pocket includes a determination regarding which noncovalent post translational modification-binding motif is recognized by the anchoring pocket. In some embodiments, the anchoring pocket is characterized as an anion anchoring pocket. In some embodiments, the anchoring pocket is characterized as a sulfation, acetylation, S-nitrosylation, methylation, proteolysis, or glycosylation anchoring pocket.

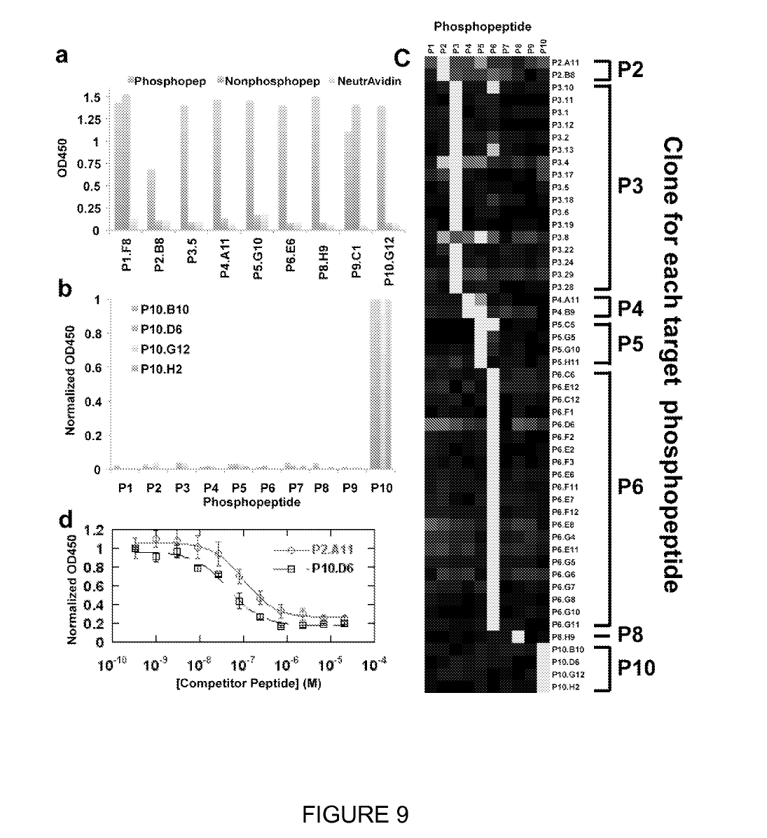

Exemplary sequences identified using the methods described herein include the following. Sequence labeling corresponds to the labeling shown in FIG. 9. CDR regions are underlined.

Listing of Antibody Sequences

>P2.A11
DIVLTQSPATLSLSPGERATLSCMTSTDIDDDMNWYQQKPGQAPRLLISEGNTLRPGVPARFSGSGSGTDFTLTIS
SLEPEDFAVYYCLQSNNIPITFGQGTKVEIKRGGGGSGGGGSGGGGSEVQLVESGGGLVQPGGSLRLSCVTSGFTF
RKFGMSWVRQAPGKGLEWVASISTPRGSTTYYSDSVKGRFTISRDNSKNTLYLQMNSLRAEDTAVYYCTRNT-----
DAW--FAYWGQGTLVTVSS
>P2.B8
DIVLTQSPATLSLSPGERATLSCMTSTDIDDDMNWYQQKPGQAPRLLISEGNTLRPGVPARFSGSGSGTDFTLTIS
SLEPEDFAVYYCLQYASYPFTFGQGTKVEIKRGGGGSGGGGSGGGGSEVQLVESGGGLVQPGGSLRLSCVTSGFTF
RKFGMSWVRQAPGKGLEWVATISTPRGSYTNYSDSVKGRFTISRDNSKNTLYLQMNSLRAEDTAVYYCTRGW-----
S----MAYWGQGTLVTVSS
>P3.29
DIVLTQSPATLSLSPGERATLSCMTSTDIDDDMNWYQQKPGQAPRLLISEGNTLRPGVPARFSGSGSGTDFTLTISS
LEPEDFAVYYCLQRYNNPITFGQGTKVEIKRGGGGSGGGGSGGGGSEVQLVESGGGLVQPGGSLRLSCVTSGFTFRK
FGMSWVRQAPGKGLEWVATISTPRGSATYYSDSVKGRFTISRDNSKNTLYLQMNSLRAEDTAVYYCTRA-G-KG-
AAFDYWGQGTLVTVSS
>P3.24
DIVLTQSPATLSLSPGERATVSCMTSTDIDDDMNWYQQKPGQAPRLLISEGNTLRPGVPARFSGSGSGTDFTLTISS
LEPEDFAVYYCLQRAAYPITFGQGTKVEIKRGGGGRGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
AGFAYWGQGTLVTVSS
>P3.22
DIVLTQSPATLSLSPGERATLSCMTSTDIDDDMNWYQQKPGQAPRLLISEGNTLRPGVPARFSGSGSGTDFTLTISS
LEPEDFAVYYCLQRYNYPITFGQGTKVEIKRGGGGSGGGGSGGGGSEVQLVESGGGLVQPGGSLRLSCVTSGFTFRK
FGMSWVRQAPGKGLEWVATISTPRGSTTYYSDSVKGRFTISRDNSKNTLYLQMNSLRAEDTAVYYCTRA-GSRG-
AAFDYWGQGTLVTVSS
>P3.18
DIVLTQSPATLSLSPGERATMSCMTSTDIDDDMNWYQQKPGQAPRLLISEGNTLRPGVPARFSGSGSGTDFTLTISS
LEPEDFAVYYCLQRNGYPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
AAMAYWGQGTLVTVSS
>P3.17
DIVLTQSPATLSLSPGERATLSCMTSTDIDDDMNWYQQKPGQAPRLLISEGNTLRPGVPARFSGSGSGTDFTLTISS
LEPEDFAVYYCLQRSGVPLTFGQGTKVEIKRGGGGSGGGGSGGGGSEVQLVESGGGLVQPGGSLRLSCVTSGFTFRK
FGMSWVRQAPGKGLEWVATISTPRGSYTNYSDSVKGRFTISRDNSKNTLYLQMNSLRAEDTAVYYCTRA-G-TG-
AAFAYWGQGTLVTVSS
>P3.13
DIVLTQSPATLSLSPGERATLSCMTSTDIDDDMNWYQQKPGQAPRLLISEGNTLRPGVPARFSGSGSGTDFTLTISS
LEPEDFAVYYCLQRAGYPVTFGQGTKVEIKRGGGGSGGGGSGGGGSEVQLVESGGGLVQPGGSLRLSCVTSGFTFRK
FGMSWVRQAPGKGLEWVATIAT-GGHTTDYSDSVKGRFTISRDNSKNTLYLQMNSLRAEDTAVYYCTRA-G-KG-
AAFAYWGQGTLVTVSS
>P3.12
DIVLTQSPATLSLSPGERATVSCMTSTDIDDDMNWYQQKPGQAPRLLISEGNTLRPGVPARFSGSGSGTDFTLTISS
LEPEDFAVYYCLQRSSYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-AG-
AGFAYWGQGTLVTVSS
>P3.11
DIVLTQSPATLSLSPGERATLSCMTSTDIDDDMNWYQQKPGQAPRLLISEGNTLRPGVPARFSGSGSGTDFTLTISS
LEPEDFAVYYCLQRSAYPITFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
GAFAYWGQGTLVTVSS
>P3.10
DIVLTQSPATLSLSPGERATLSCMTSTDIDDDMNWYQQKPGQAPRLLISEGNTLRPGVPARFSGSGSGTDFTLTISS
LEPEDFAVYYCLQRTGYPITFGQGTKVEIKRGGGGSGGGGSGGGGSEVQLVESGGGLVQPGGSLRLSCVTSGFTFRK
FGMSWVRQAPGKGLEWVATISTPRGSSTNYSDSVKGRFTISRDNSKNTLYLQMNSLRAEDTAVYYCTRA-G-DG-
AAFDYWGQGTLVTVSS
>P3.6
DIVLTQSPATLSLSPGERATLSCMTSTDIDDDMNWYQQKPGQAPRLLISEGNTLRPGVPARFSGSGSGTDFTLTISS
LEPEDFAVYYCLQRAGFPLTFGQGTKVEIKRGGGGSGGGGSGGGGSEVQLVESGGGLVQPGGSLRLSCVTSGFTFRK
FGMSWVRQAPGKGLEWVATIAT-GGHTTNYSDSVKGRFTISRDNSKNTLYLQMNSLRAEDTAVYYCTRA-G-
AGGAAFDYWGQGTLVTVSS
>P3.5
DIVLTQSPATLSLSPGERATLSCMTSTDIDDDMNWYQQKPGQAPRLLISEGNTLRPGVPARFSGSGSGTDFTLTISS
LEPEDFAVYYCLQRSAFPLTFGQGTKVEIKRGGGGSGGGGSGGGGSEVQLVESGGGLVQPGGSLRLSCVTSGFTFRK
FGMSWVRQAPGKGLEWVASISTPRGSDTDYSDSVKGRFTISRDNSKNTLYLQMNSLRAEDTAVYYCTRA-G-AG-
SSFDYWGQGTLVTVSS
>P3.4
DIVLTQSPATLSLSPGERATLSCMTSTDIDDDMNWYQQKPGQAPRLLISEGNTLRPGVPARFSGSGSGTDFTLTISS
LEPEDFAVYYCLQRATYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-AG-
TAFDYWGQGTLVTVSS
>P3.1
DIVLTQSPATLSLSPGERATLSCMTSTDIDDDMNWYQQKPGQAPRLLISEGNTLRPGVPARFSGSGSGTDFTLTISS
LEPEDFAVYYCLQRSAFPITFGQGTKVEIKRGGGGSGGGGSGGGGSEVQLVESGGGLVQPGGSLRLSCVTSGFTFRK
FGMSWVRQAPGKGLEWVATISTPRGSTTDYSDSVKGRFTISRDNSKNTLYLQMNSLRAEDTAVYYCTRT-A-AG-
AAFAYWGQGTLVTVSS
>P3.28
DIVLTQSPATLSLSPGERATLSCMTSTDIDDDMNWYQQKPGQAPRLLISEGNTLRPGVPARFSGSGSGTDFTLTISS
LEPEDFAVYYCLQRNAYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-NG-
AAMAYWGQGTLVTVSS
>P3.19
DIVLTQSPATLSLSPGERATVSCMTSTDIDDDMNWYQQKPGQAPRLLISEGNTLRPGVPARFSGSGSGTDFTLTISS
LEPEDFAVYYCLQHYSFPITFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRGAR-RG-
EGFDYWGQGTLVTVSS
>P3.8
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRGKG----
KKMDYWGQGTLVTVSS
>P3.2
DIVLTQSPATLSLSPGERATLSCMTSTDIDDDMNWYQQKPGQAPRLLISEGNTLRPGVPARFSGSGSGTDFTLTISS
LEPEDFAVYYCLQRSAYPITFGQGTKVEIKRGGGGSGGGGSGGGGSEVQLVESGGGLVQPGGSLRLSCVTSGFTFRK
FGMSWVRQAPGKGLEWVASIAT-GGHTTNYSDSVKGRFTISRDNSKNTLYLQMNSLRAEDTAVYYCTRA-G-GG-
ASFAYWGQGTLVTVSS
>P4.B9
DIVLTQSPATLSLSPGERATLSCMTSTDIDDDMNWYQQKPGQAPRLLISEGNTLRPGVPARFSGSGSGTDFTLTIS
SLEPEDFAVYYCLQGTNDPVTFGQGTKVEIKRGGGGSGGGGSGGGGSEVQLVESGGGLVQPGGSLRLSCVTSGFTF
RKFGMSWVRQAPGKGLEWVASISTPRGSSTYYSDSVKGRFTISRDNSKNTLYLQMNSLRAEDTAVYYCTRGYSSTS
YAMDYWGQGTLVTVSS
>P4.A11
DIVLTQSPATLSLSPGERATLSCMTSTDIDDDMNWYQQKPGQAPRLLISEGNTLRPGVPARFSGSGSGTDFTLTIS
SLEPEDFAVYYCLQRNAVPFTFGQGTKVEIKRGGGGSGGGGSGGGGSEVQLVESGGGLVQPGGSLRLSCVTSGFTF
RKFGMSWVRQAPGKGLEWVAGISTPRGSYTDYSDSVKGRFTISRDNSKNTLYLQMNSLRAEDTAVYYCTRG-GGAG
AGFDYWGQGTLVTVSS
>P5.G5
DIVLTQSPATLSLSPGERATLSCMTSTDIDDDMNWYQQKPGQAPRLLISEGNTLRPGVPARFSGSGSGTDFTLTISS
LEPEDFAVYYCLQHYDIPLTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------GKA--
MDYWGQGTLVTVSS
>P5.H11
DIVLTQSPATLSLSPGERATLSCMTSTDIDDDMNWYQQKPGQAPRLLISEGNTLRPGVPARFSGSGSGTDFTLTISS
LEPEDFAVYYCLQSDSFPVTFGQGTKVEIKRGGGGSGGGGSGGGGSEVQLVESGGGLVQPGGSLRLSCVTSGFTFRK
FGMSWVRQAPGKGLEWVATIATGGHTTYYSDSVKGRFTISRDNSKNTLYLQMNSLRAEDTAVYYCTRWSWDNRSAT--
MDYWGQGTLVTVSS
>P5.G10
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------
STAAWFDYWGQGTLVTVSS
>P5.C5
DIVLTQSPATLSLSPGERATLSCMTSTDIDDDMNWYQQKPGQAPRLLISEGNTLRPGVPARFSGSGSGTDFTLTISS
LEPEDFAVYYCLQYDAFPFTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------GGE-
MDYWGQGTLVTVSS
>P6.G11
DIVLTQSPATLSLSPGERATLSCMTSTDIDDDMNWYQQKPGQAPRLLISEGNTLRPGVPARFSGSGSGTDFTLTISS
LEPEDFAVYYCLQYSDLPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
E--G---FAYWGQGTLVTVSS
>P6.G10
DIVLTQSPATLSLSPGERATLSCMTSTDIDDDMNWYQQKPGQAPRLLISEGNTLRPGVPARFSGSGSGTDFTLTISS
LEPEDFAVYYCLQDASFPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----ATTT
S--T-----FAYWGQGTLVTVSS
>P6.G8
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----A---
E--N-----MDYWGQGTLVTVSS
>P6.G7
DIVLTQSPATLSLSPGERATLSCMTSTDIDDDMNWYQQKPGQAPRLLISEGNTLRPGVPARFSGSGSGTDFTLTISS
LEPEDFAVYYCLQYNGIPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
T--G-----FDYWGQGTLVTVSS
>P6.G6
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
A--S-----MDYWGQGTLVTVSS
>P6.G5
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
E--S-----MDYWGQGTLVTVSS
>P6.G4
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
GYYA-----FDYWGQGTLVTVSS
>P6.F12
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
A--S-----MDYWGQGTLVTVSS
>P6.F11
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTR-----T---
N--N--SSWFDYWGQGTLVTVSS
>P6.F3
DIVLTQSPATLSLSPGERATLSCMTSTDIDDDMNWYQQKPGQAPRLLISEGNTLRPGVPARFSGSGSGTDFTLTISS
LEPEDFAVYYCLQYAGVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
S--A-----MDYWGQGTLVTVYS
>P6.F2
DIVLTQSPATLSLSPGERATLSCMTSTDIDDDMNWYQQKPGQAPRLLISEGNTLRPGVPARFSGSGSGTDFTLTISS
LEPEDFAVYYCLQYAGVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
A--T-----MDYWGQGTLVTVSS
>P6.F1
DIVLTQSPATLSLSPGERATLSCMTSTDIDDDMNWYQQKPGQAPRLLISEGNTLRPGVPARFSGSGSGTDFTLTISS
LEPEDFAVYYCLQGDAIPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----D---
S--G-----MDYWGQGTLVTVSS
>P6.E12
DIVLTQSPATLSLSPGERATLSCMTSTDIDDDMNWYQQKPGQAPRLLISEGNTLRPGVPARFSGSGSGTDFTLTISS
LEPEDFAVYYCLQHYNVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
Y--G-----FDYWGQGTLVTVSS
>P6.E11
DIVLTQSPATLSLSPGERATLSCMTSTDIDDDMNWYQQKPGQAPRLLISEGNTLRPGVPARFSGSGSGTDFTLTISS
LEPEDFAVYYCLQYADIPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
G--S-----MDYWGQGTLVTVSS
>P6.E8
DIVLTQSPATLSLSPGERATLSCMTSTDIDDDMNWYQQKPGQAPRLLISEGNTLRPGVPARFSGSGSGTDFTLTISS
MEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
S--T-----FDYWGQGTLVTVSS
>P6.E7
DIVLTQSPATLSLSPGERATLSCMTSTDIDDDMNWYQQKPGQAPRLLISEGNTLRPGVPARFSGSGSGTDFTLTISS
LEPEDFAVYYCLQYTSVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----A---
D--E-----MDYWGQGTLVTVSS
>P6.E6
DIVLTQSPATLSLSPGERATLSCMTSTDIDDDMNWYQQKPGQAPRLLISEGNTLRPGVPARFSGSGSGTDFTLTISS
LEPEDFGVYYCLQDYGFPVTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
S--N--KDWFDYWGQGTLVTVSS
>P6.E2
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----K---
S--T-----FDYWGQGTLVTVSS
>P6.D6
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVAAIAT-GGHTTYYSDSVKGRFTISRDNSKNTLYLQMNSLRAEDTAVYYCTRSYYS-----
G--S-----MDYWGQGTLVTVSS
>P6.C12
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----R---
A--N-----FDYWGQGTLVTVSS
>P6.C6
DIVLTQSPATLSLSPGERATLSCMTSTDIDDDMNWYQQKPGQAPRLLISEGNTLRPGVPARFSGSGSGTDFTLTISS
LEPEDFAVYYCLQSASIPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
E--A-----MDYWGQGTLVTVSS
>P10.G12
DIVLTQSPATLSLSPGERATLSCMTSTDIDDDMNWYQQKPGQAPRLLISEGNTLRPGVPARFSGSGSGTDFTLTIS
SLEPEDFAVYYCLQYAGLPLTFGQGTKVEIKRGGGGSGGGGSGGGGSEVQLVESGGGLVQPGGSLRLSCVTSGFTF
RKFGMSWVRQAPGKGLEWVAGISTPRGSNTYYSDSVKGRFTISRDNSKNTLYLQMNSLRAEDTAVYYCTRAGRGEG-
FAYWGQGTLVTVSS
>P10.D6
DIVLTQSPATLSLSPGERATLSCMTSTDIDDDMNWYQQKPGQAPRLLISEGNTLRPGVPARFSGSGSGTDFTLTIS
SLEPEDFAVYYCLQHATVPFTFGQGTKVEIKRGGGGSGGGGSGGGGSEVQLVESGGGLVQPGGSLRLSCVTSGFTF
RKFGMSWVRQAPGKGLEWVASISTPRGSTTYYSDSVKGRFTISRDNSKNTLYLQMNSLRAEDTAVYYCTRTT-WNN
YFAYWGQGTLVTVSS
>P10.B10
DIVLTQSPATLSLSPGERATLSCMTSTDIDDDMNWYQQKPGQAPRLLISEGNTLRPGVPARFSGSGSGTDFTLTIS
SLEPEDFAVYYCLQHNTFPFTFGQGTKVEIKRGGGGSGGGGSGGGGSEVQLVESGGGLVQPGGSLRLSCVTSGFTF
RKFGMSWVRQAPGKGLEWVARISTPRGSNTDYSDSVKGRFTISRDNSKNTLYLQMNSLRAEDTAVYYCTRGG-AYN-
FAYWGQGTLVTVSS
>P10.H2
DIVLTQSPATLSLSPGERATLSCMTSTDIDDDMNWYQQKPGQAPRLLISEGNTLRPGVPARFSGSGSGTDFTLTIS
SLEPEDFAVYYCLQGSGAPFTFGQGTKVEIKRGGGGSGGGGSGGGGSEVQLVESGGGLVQPGGSLRLSCVTSGFTF
RKFGMSWVRQAPGKGLEWVAEIAT-GGHTTYYSDSVKGRFTISRDNSKNTLYLQMNSLRAEDTAVYYCTRGG-SMD
---YWGQGTLVTVSS
>pSAb_HC (Heavy Chain)
EISEVQLVESGGGLVQPGGSLRLSCVTSGFTFRKFGMSWVRQAPGKGLEWVASIAT-GGHTTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pSTAb_HC (Heavy Chain)
EISEVQLVESGGGLVQPGGSLRLSCVTSGFTFRKFGMSWVRQAPGKGLEWVASISTPRGSTTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pYAb_HC (Heavy Chain)
EISEVQLVESGGGLVQPGGSLRLSCVTSGFTFRKFGMSWVRQAPGKGLEWVASIVG--GRKTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pSAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
>pSTAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
>pYAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
ID P8.H9 STANDARD;
PRT; 241 AA
SQ Sequence 241 AA;

| | |
|---|---|
| DIVLTQSPAT LSLSPGERAT LSCMTSTDID DDMNWYQQKP GQAPRLLISE GNTLRPGVPA | 60 |
| RFSGSGSGTD FTLTISSLEP EDFAVYYCLQ STGVPFTFGQ GTKVEIKRGG GGSGGGGSGG | 120 |
| GGSEVQLVES GGGLVQPGGS LRLSCVTSGF TFRKFGMSWV RQAPGKGLEW VASIATGGHT | 180 |
| TYYSDSVKGR FTISRDNSKN TLYLQMNSLR AEDTAVYYCT RNSSDAWMAY WGQGTLVTVS | 240 |
| S | 241 |

In some embodiments, the antibodies indentified by the methods disclosed herein include comprise one or more CDRs as part of the anchoring pocket. Exemplary CDR sequences include but are not limited to those listed in Table 2 below. In some embodiments, the CDRs comprising the anchoring pocket include one or more CDRs as listed in Table 2. In some embodiments, the CDRs comprising the anchoring pocket include 1, 2, 3, 4, 5, 6 or more CDRs as listed in Table 2. In some embodiments, the CDRs comprising the anchoring pocket bind a phosphorylated residue. In some embodiments, the CDRs comprising the anchoring pocket include a H2 CDR2 as listed in Table 2.

TABLE 2
Exemplary CDR sequences

| Antibody Designation | L1 CDR1 | L2 CDR2 | L3 CDR3 | H1 CDR1 | H2 CDR2 | H3 CDR3 |
|---|---|---|---|---|---|---|
| >P2.A11 | MTSTDIDDDMN | EGNTLRP | LQSNNIPI | GFTFRKFGMS | SISTPRGSTTY | TRNTDAWFAY |
| >P2.B8 | MTSTDIDDDMN | EGNTLRP | LQYASYPF | GFTFRKFGMS | TISTPRGSYTN | TRGWSMAY |
| >P3.29 | MTSTDIDDDMN | EGNTLRP | LQRYNNPI | GFTFRKFGMS | TISTPRGSATY | TRAGKGAAFDY |
| >P3.24 | MTSTDIDDDMN | EGNTLRP | LQRAAYPI | GFTFRKFGMS | SIATGGHTTY | TRAGAGAGFAY |
| >P3.22 | MTSTDIDDDMN | EGNTLRP | LQRYNYPI | GFTFRKFGMS | TISTPRGSTTY | TRAGSRGAAFDY |
| >P3.18 | MTSTDIDDDMN | EGNTLRP | LQRNGYPL | GFTFRKFGMS | SIATGGHTTY | TRAGAGAAMAY |
| >P3.17 | MTSTDIDDDMN | EGNTLRP | LQRSGVPL | GFTFRKFGMS | TISTPRGSYTN | TRAGTGAAFAY |
| >P3.13 | MTSTDIDDDMN | EGNTLRP | LQRAGYPV | GFTFRKFGMS | TIATGGHTTD | TRAGKGAAFAY |
| >P3.12 | MTSTDIDDDMN | EGNTLRP | LQRSSYPL | GFTFRKFGMS | SISTPRGSTTY | TRAGAGAGFAY |
| >P3.11 | MTSTDIDDDMN | EGNTLRP | LQRSAYPI | GFTFRKFGMS | SIATGGHTTY | TRAGAGGAFAY |
| >P3.10 | MTSTDIDDDMN | EGNTLRP | LQRTGYPI | GFTFRKFGMS | TISTPRGSSTN | TRAGDGAAFDY |

| | | | | | | |
|---|---|---|---|---|---|---|
| >P3.6 | MTSTDIDDDMN | EGNTLRP | LQRAGFPL | GFTFRKFGMS | TIATGGHTTN | TRAGAGGAAFDY |

| | | | | | | |
|---|---|---|---|---|---|---|
| >P3.5 | MTSTDIDDDMN | EGNTLRP | LQRSAFPL | GFTFRKFGMS | SISTPRGSDTD | TRAGAGSSFDY |
| >P3.4 | MTSTDIDDDMN | EGNTLRP | LQRATYPL | GFTFRKFGMS | SISTPRGSTTY | TRAGAGTAFDY |
| >P3.1 | MTSTDIDDDMN | EGNTLRP | LQRSAFPI | GFTFRKFGMS | TISTPRGSTTD | TRTAAGAAFAY |
| >P3.28 | MTSTDIDDDMN | EGNTLRP | LQRNAYPL | GFTFRKFGMS | SISTPRGSTTY | TRAGNGAAMAY |
| >P3.19 | MTSTDIDDDMN | EGNTLRP | LQHYSFPI | GFTFRKFGMS | SISTPRGSTTY | TRGARRGEGFDY |
| >P3.8 | MTSTDIDDDMN | EGNTLRP | LQSFNVPL | GFTFRKFGMS | SIATGGHTTY | TRGKGKKMDY |
| >P3.2 | MTSTDIDDDMN | EGNTLRP | LQRSAYPI | GFTFRKFGMS | SIATGGHTTN | TRAGGGASFAY |
| >P4.B9 | MTSTDIDDDMN | EGNTLRP | LQGTNDPV | GFTFRKFGMS | SISTPRGSSTY | TRGYSSTSYAMDY |
| >P4.A11 | MTSTDIDDDMN | EGNTLRP | LQRNAVPF | GFTFRKFGMS | GISTPRGSYTD | TRGGGAGAGFDY |
| >P5.G5 | MTSTDIDDDMN | EGNTLRP | LQHYDIPL | GFTFRKFGMS | SIATGGHTTY | TRGKAMDY |
| >P5.H11 | MTSTDIDDDMN | EGNTLRP | LQSDSFPV | GFTFRKFGMS | TIATGGHTTY | TRWSWDNRSATMDY |
| >P5.G10 | MTSTDIDDDMN | EGNTLRP | LQSFNVPL | GFTFRKFGMS | SIATGGHTTY | TRSTAAWFDY |
| >P5.C5 | MTSTDIDDDMN | EGNTLRP | LQYDAFPF | GFTFRKFGMS | SIATGGHTTY | TRGGE-MDY |
| >P6.G11 | MTSTDIDDDMN | EGNTLRP | LQYSDLPF | GFTFRKFGMS | SIATGGHTTY | TRGEGFAY |
| >P6.G10 | MTSTDIDDDMN | EGNTLRP | LQDASFPL | GFTFRKFGMS | SIATGGHTTY | TRATTTSTFAY |
| >P6.G8 | MTSTDIDDDMN | EGNTLRP | LQSFNVPL | GFTFRKFGMS | SIATGGHTTY | TRAENMDY |
| >P6.G7 | MTSTDIDDDMN | EGNTLRP | LQYNGIPF | GFTFRKFGMS | SIATGGHTTY | TRSTGFDY |
| >P6.G6 | MTSTDIDDDMN | EGNTLRP | LQSFNVPL | GFTFRKFGMS | SIATGGHTTY | TRGASMDY |
| >P6.G5 | MTSTDIDDDMN | EGNTLRP | LQSFNVPL | GFTFRKFGMS | SIATGGHTTY | TRSESMDY |
| >P6.G4 | MTSTDIDDDMN | EGNTLRP | LQSFNVPL | GFTFRKFGMS | SIATGGHTTY | TRSGYYAFDY |
| >P6.F12 | MTSTDIDDDMN | EGNTLRP | LQSFNVPL | GFTFRKFGMS | SIATGGHTTY | TRNASMDY |
| >P6.F11 | MTSTDIDDDMN | EGNTLRP | LQSFNVPL | GFTFRKFGMS | SISTPRGSTTY | TRTNNSSWFDY |
| >P6.F3 | MTSTDIDDDMN | EGNTLRP | LQYAGVPF | GFTFRKFGMS | SIATGGHTTY | TRNSAMDY |
| >P6.F2 | MTSTDIDDDMN | EGNTLRP | LQYAGVPL | GFTFRKFGMS | SIATGGHTTY | TRNATMDY |
| >P6.F1 | MTSTDIDDDMN | EGNTLRP | LQGDAIPF | GFTFRKFGMS | SIATGGHTTY | TRDSGMDY |
| >P6.E12 | MTSTDIDDDMN | EGNTLRP | LQHYNVPF | GFTFRKFGMS | SIATGGHTTY | TRSYGFDY |
| >P6.E11 | MTSTDIDDDMN | EGNTLRP | LQYADIPL | GFTFRKFGMS | SIATGGHTTY | TRGGSMDY |
| >P6.E8 | MTSTDIDDDMN | EGNTLRP | LQSFNVPL | GFTFRKFGMS | SIATGGHTTY | TRGSTFDY |
| >P6.E7 | MTSTDIDDDMN | EGNTLRP | LQYTSVPF | GFTFRKFGMS | SIATGGHTTY | TRADEMDY |
| >P6.E6 | MTSTDIDDDMN | EGNTLRP | LQDYGFPV | GFTFRKFGMS | SIATGGHTTY | TRSSNKDWFDY |
| >P6.E2 | MTSTDIDDDMN | EGNTLRP | LQSFNVPL | GFTFRKFGMS | SIATGGHTTY | TRKSTFDY |
| >P6.D6 | MTSTDIDDDMN | EGNTLRP | LQSFNVPL | GFTFRKFGMS | AIATGGHTTY | TRSYYSGSMDY |
| >P6.C12 | MTSTDIDDDMN | EGNTLRP | LQSFNVPL | GFTFRKFGMS | SIATGGHTTY | TRRANFDY |
| >P6.C6 | MTSTDIDDDMN | EGNTLRP | LQSASIPL | GFTFRKFGMS | SIATGGHTTY | TRGEAMDY |
| >P10.G12 | MTSTDIDDDMN | EGNTLRP | LQYAGLPL | GFTFRKFGMS | GISTPRGSNTY | TRAGRGEGFAY |
| >P10.D6 | MTSTDIDDDMN | EGNTLRP | LQHATVPF | GFTFRKFGMS | SISTPRGSTTY | TRTTWNNYFAY |
| >P10.B10 | MTSTDIDDDMN | EGNTLRP | LQHNTFPF | GFTFRKFGMS | RISTPRGSNTD | TRGGAYNFAY |
| >P10.H2 | MTSTDIDDDMN | EGNTLRP | LQGSGAPF | GFTFRKFGMS | EIATGGHTTY | TRGGSMDY |
| >pSAb_HC (Heavy Chain) | ----------- | ---------- | --------- | GFTFRKFGMS | SIATGGHTTY | TRGYSSTSYAMDY |
| >pSTAb_HC (Heavy Chain) | ----------- | ---------- | --------- | GFTFRKFGMS | SISTPRGSTTY | TRGYSSTSYAMDY |
| >pYAb_HC (Heavy Chain) | ----------- | ---------- | --------- | GFTFRKFGMS | SIVGGRKTY | TRGYSSTSYAMDY |
| >pSAb_LC (Light Chain) | MTSTDIDDDMN | EGNTLRP | LQSFNVPL | ----------- | ----------- | ----------- |
| >pSTAb_LC (Light Chain) | MTSTDIDDDMN | EGNTLRP | LQSFNVPL | ----------- | ----------- | ----------- |
| >pYAb_LC (Light Chain) | MTSTDIDDDMN | EGNTLRP | LQSFNVPL | ----------- | ----------- | ----------- |
| >ID 8.H9 (STANDARD) | MTSTDIDDDMN | EGNTLRP | LQSTGVPF | GFTFRKFGMS | SIATGGHTTY | TRNSSDAWMAY |

### EXAMPLES

The methods system herein described are further illustrated in the following examples, which are provided by way of illustration and are not intended to be limiting.

### Example 1

### Design of Phospho-Specific Antibody Scaffolds

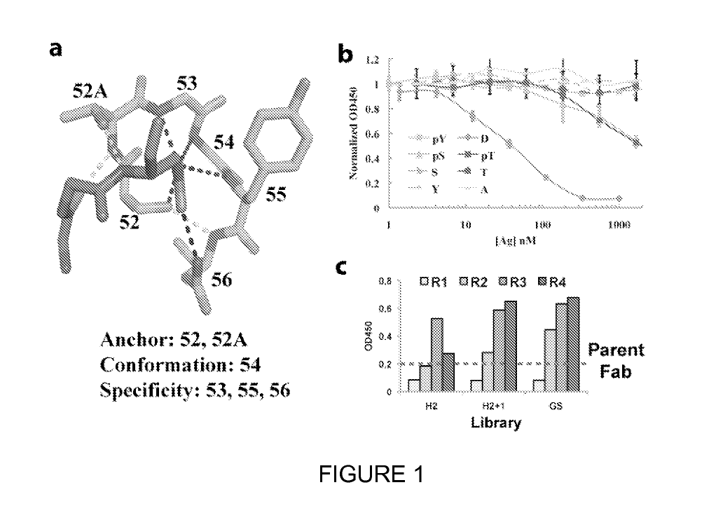

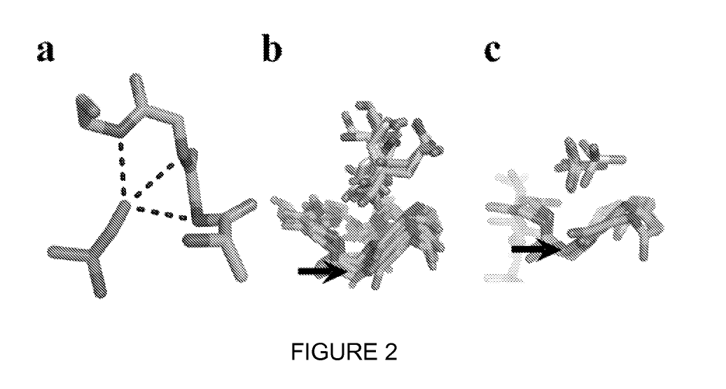

The most common anion-binding motif within many different protein superfamilies, such as ATPases, helicases, and kinases, consists of three consecutive residues where multiple main-chain amides form hydrogen bonds with the anion (FIG. 1a) (Watson, J. D. & Milner-White, E. J. A novel main-chain anion-binding site in proteins: the nest. A particular combination of phi,psi values in successive residues gives rise to anion-binding sites that occur commonly and are found often at functionally important regions. J Mol Biol 315, 171-182 (2002)). An existing Ab scaffold into which a similar loop could be built was sought. The CDRs were manually inspected for the desired nest conformation from sixty anti-peptide Ab structures. A region of CDR H2 within a mouse Fab (PDB ID 1i8i) (Landry, R. C. et al. Antibody recognition of a conformational epitope in a peptide antigen: Fv-peptide complex of an antibody fragment specific for the mutant EGF receptor, EGFRvIII. J Mol Biol 308, 883-893 (2001)) was identified that adopts the desired conformation due a hallmark α_{L} glycine at 54_{H} (FIG. 1b). This Ab utilized the H2 loop to bind an acidic residue, which is often used as a phospho-mimic, in the peptide. This recognition is achieved by six loop residues that anchor the peptide (52_{H} and 52A_{H}), stabilize the conformation (54_{H}), or confer specificity to the Asp side chain (53_{H}, 55_{H}, and 56_{H}) (FIG. 1a and Table 3). A larger search of all Ab-antigen structures identified eight Abs that utilize this loop to make hydrogen bonds between the main-chain NH groups in the Ab and an aspartate or glutamate in the peptide (FIG. 2b).

TABLE 3
Functional description of H2 loop residues.

| Heavy chain | | |
|---|---|---|
| residue | Label | Function |
| 52 | Anchor | Accepts hydrogen bond from main-chain |
| | | amide of 56; Donates hydrogen bond to |
| | | carboxylate of Asp |
| 52A | Anchor | Hydrogen bonds to main-chain amide of |
| | | Asp; Potential hydrogen bond to |
| | | phosphate |
| 53 | Specificity | Lack of side chain prevents steric |
| | | clashes with Asp |
| 54 | Conformation | Critical α_{L} glycine |
| 55 | Specificity | Side chain can confer specificity and |
| | | enhance binding |
| 56 | Specificity | Side chain can confer specificity and |
| | | enhance binding |

To characterize this class of Ab-antigen interactions, gene encoding the mouse 1i8i Fab was synthesized and cloned into a phage display vector and a protein expression vector (Table 4). A humanized version of the Fab by grafting the six CDRs onto a robustly expressing human Fab scaffold was developed. This humanized scaffold, which expressed at yields >3 mg/L in bacteria, bound the peptide with similar affinity as reported for the mouse Fab (Landry, R. C. et al. Antibody recognition of a conformational epitope in a peptide antigen: Fv-peptide complex of an antibody fragment specific for the mutant EGF receptor, EGFRvIII. J Mol Biol 308, 883-893 (2001)). This suggests that the peptide-binding site was preserved between the mouse and humanized Fabs, which was confirmed by subsequent crystallographic analysis of the humanized Ab.

TABLE 4
List of vectors utilized.

| Vector | Type | Promoter | Description |
|---|---|---|---|
| pJK1 | Phagemid with | phoA | Displayed protein is fused to |
| | truncated g3 | | C-terminal domain of g3 |
| pJK2 | Phagemid with | phoA | Display protein is fused to |
| | full-length g3 | | full-length domain of g3 |
| pJK3 | Protein expression | T7 | Expression under control of |
| | in bacteria | | T7 promoter |
| pJK4 | Protein expression | pTac | Expression under control of |
| | in bacteria | | pTac promoter |
| pJK5 | Protein expression | T7 | Expression under control of |
| | in bacteria | | T7; co-expression of BirA |
| pJK6 | Protein expression | hEFI- | Mammalian cell expression of |
| | in mammalian cells | HTLV | protein fused to rabbit Fc |

To understand the importance of the Asp-loop (residues 50_{H}-56_{H}) interaction in peptide binding, the humanized Fab was displayed on bacteriophage and competition ELISAs were performed to analyze binding of the humanized Fab to a panel of peptides. ELISA data confirmed that the Asp8 residue of the antigen is a hot spot for binding as mutation to Ala, Ser, Thr, or Tyr substantially reduced Ab binding (>100-fold less) to the peptide (FIG. 1c). Without being bound by theory, the carboxylate group of Asp8 residue might mimic a phosphorylated residue and thus, the Ab can bind peptides with pSer, pThr, or possibly pTyr in place of Asp8 (Table 5). ELISA data confirmed the ability of this Fab to bind pSer- or pThr-containing peptides, albeit with weak affinities (>2000 nM) (FIG. 1c and Table 6).

TABLE 5
Phosphorylated peptide antigens utilized.

| Antigen | Peptide sequence |
|---|---|
| pSer | Biotin-GEKKGNYVVTpSH |
| pThr | Biotin-GEKKGNYVVTpTH |
| pTyr | Biotin-GEKKGNYVVTpYA |
| Caspase 3 (S12) | Biotin-NTENSVDSKpSIKNLEPKII |
| RIPK3 (S227) | Biotin-REVELPTEPpSLVYEAV |
| RIPK3 (S199) | Biotin-LFVNVNRKApST ASDVYSF |
| Smad2 (T8) | Biotin-MSSILPFpTPPVVKRLL |
| CREB (S133) | Biotin-RREILSRRPpSYTKILNDL |
| HtrA2 (S212) | Biotin-RRRVRVRLLpSGDTYEAVV |
| Akt1 (T308) | Biotin-KEGIKDGATMKpTF |
| Akt1 (S473) | Biotin-ERRPHFPQFpSYSASGTA |
| PKC theta (S695) | Biotin-DQNMFRNFpSFMNPGMER |
| Sgk1 (S422) | Biotin-EAAEAFLGFpSYAPPTDSF |

TABLE 6
Affinity measurements of antibody scaffolds.

| Fab | Peptide | kₒₙ (M⁻¹ s⁻¹) | k_{off} (s⁻¹) | K_{D} (nM) |
|---|---|---|---|---|
| Parent | WT Asp | 3.38 × 10⁵ | 0.0032 | 9.6 |
| | pSer | n.d. | n.d. | >2000^{a} |
| | pThr | n.d. | n.d. | >2000^{a} |
| | Ser/Thr | n.d. | n.d. | >2000^{a} |
| pSAb | pSer | 1.0 × 10⁵ | 0.0075 | 71 |
| | pThr | 4.7 × 10⁴ | 0.041 | 866 |
| | Ser/Thr | n.d. | n.d. | >2000^{a} |
| pSTAb | pSer | 4.8 × 10⁴ | 0.0082 | 172 |
| | pThr | 2.8 × 10⁴ | 0.0064 | 232 |
| | Ser/Thr | n.d. | n.d. | >2000^{a} |
| pYAb | pTyr | 1.9 × 10⁵ | 0.070 | 360 |
| | Tyr | 2.84 × 10⁴ | 0.249 | 8700 |

^{a}No binding seen by competition ELISAs.

To optimize the CDR for binding to each phosphorylated residue, three Ab phage display libraries were constructed (all with diversities exceeding 5×10⁹). The six-residue CDR region (52_{H}-56_{H}) was replaced with six fully random residues (H2 library) or seven fully random residues (H2+1 library) to relieve steric clashes with the Ab backbone for each of the peptides. The third library design was similar to the H2 library, but fixed Gly or Ser at 53_{H} and 54_{H} (GS library). This strategy permitted the assessment of the importance of the anchor (52_{H} and 52A_{H}) and conformation (55_{H}) residues as well as altering the specificity residues (53_{H}, 55_{H}, and 56_{H}). Using standard phage display methods, four rounds of selection against the pSer, pThr, or pTyr peptides were performed. Strong enrichment against each of pSer, pThr, and pTyr peptide targets was observed using all three libraries, except for selections with the H2+1 library against pTyr (FIG. 1c).

### Vector Construction

A series of p3 phage display vectors along with compatible protein expression vectors was constructed (Table 4). A previously described phagemid was designed to express a Fab from a phoA promoter and display the Fab on gene 3 of M13 bacteriophage (Sidhu, S. S. et al. Phage-displayed antibody libraries of synthetic heavy chain complementarity determining regions. J Mol Biol 338, 299-310 (2004)). In place of the original Fab gene, a cassette was inserted having of a Pe1B signal sequence followed by a dummy gene sequence, which is then linked to a truncated gene III coat protein (pJK1). Additionally, a version with a full-length gene III coat protein (pJK2) along with two protein expression vectors was constructed (pJK3 and pJK4). To permit efficient cloning of antibody sequences between all vectors, a dummy gene was flanked by two unique Sfi I restriction sites. To construct the initial Fab scaffold of 1i8i, a gene cassette encoding the heavy and light chains of the mouse Fab was synthesized and cloned into pJK1. A humanized version of this scaffold was also constructed by grafting the three heavy chain CDRs from the mouse Fab onto a consensus VH3 heavy chain gene and the three light chain CDRs onto a consensus VLK3 light chain gene (Knappik, A. et al. Fully synthetic human combinatorial antibody libraries (HuCAL) based on modular consensus frameworks and CDRs randomized with trinucleotides. J Mol Biol 296, 57-86 (2000)). The human Fab template was modified by Kunkel mutagenesis, according to standard protocols (Kunkel, T. A. Rapid and efficient site-specific mutagenesis without phenotypic selection. Proc Natl Acad Sci USA 82, 488-492 (1985)). All restriction enzymes and DNA polymerases were purchased from NEB (Ipswich, Mass.). Oligonucleotides were purchased from IDT and all constructs were verified by DNA sequencing (Quintara Biosciences).

### Generation of Phage Libraries

A humanized Fab in pJK1 with two stop codons within the CDR H2 was used as a template for Kunkel mutagenesis with oligonucleotides designed to correct the stop codons and introduce the designed mutations at each site (Sidhu, S. S. et al. Phage-displayed antibody libraries of synthetic heavy chain complementarity determining regions. J Mol Biol 338, 299-310 (2004) and Kunkel, T. A. Rapid and efficient site-specific mutagenesis without phenotypic selection. Proc Natl Acad Sci USA 82, 488-492 (1985)). To make the H2-targeted libraries, a set of three libraries was generated in which the codons encoding the parent H2 sequence (STGGYN) was replaced with either i) six random amino acids encoded by NNK (H2 library), ii) seven random amino acids encoded by NNK (H2+1 library), or iii) a core set of two or three amino acids, which were allowed to be only Gly or Ser, and were flanked on both sides by two random amino acids encoded by NNK (GS library). Mutagenic oligonucleotides are listed in Table 7. The resulting mutagenesis reactions were electroporated and phage were produced as previously described (Sidhu, S. S. et al. Phage-displayed antibody libraries of synthetic heavy chain complementarity determining regions. J Mol Biol 338, 299-310 (2004)). The final diversities of the H2, H2+1, and GS libraries were 6.5×10⁹, 1.6×10¹⁰, and 5.3×10⁹, respectively.

TABLE 7
Oligonucleotides used to generate antibody libraries

| Name | Sequence |
|---|---|
| H2 NNK | GGAATGGGTTGCATCCATTNNKNNKNNKNNKNNKNNKACCTACTATAGCGATAGCGT |
| H2+1 NNK | GGATGGGTTGCATCCATTNNKNNKNNKNNKNNKNNKNNKACCTACTATAGCGATAGCGT |
| GS2 NNK | GGAATGGGTTGCATCCATTNNKNNKRGCRGCNNKNNKACCTACTATAGCGATAGCGT |
| GS3 NNK | GGAATGGGTTGCATCCATTNNKNNKRGCRGCNNKNNKACCTACTATAGCGATAGCGT |
| L3 P1 | GCGGTGTATTATTGCCTTCAATMTDMCRVTNHTCCCNTTACCTTTGGACAGggtacc |
| L3 P2 | GCGGTGTATTATTGCCTTCAASRTDMCRVTNHTCCCNTTACCTTTGGACAGggtacc |
| H2 S1 | GCCTGGAATGGGTTGCAGAAATTgcgACtGGCGGCCATaccACCDACTATAGCGATAGCGTCAAGGG |
| H2 S2 | GCCTGGAATGGGTTGCADGGATTgcgACtGGCGGCCATaccACCDACTATAGCGATAGCGTCAAGGG |
| H2 S3 | GCCTGGAATGGGTTGCADHTATTgcgACtGGCGGCCATaccACCDACTATAGCGATAGCGTCAAGGG |
| H2 ST1 | GCCTGGAATGGGTTGCAGAAATTAGCACC CCCCGCGGGTCTDMTACCDACTATAGDGATAGCGTCAAGGG |
| H2 ST2 | GCCTGGAATGGGTTGCADGGATTAGCACC CCCCGCGGGTCTDMTACCDACTATAGCGATAGCGTCAAGGG |
| H2 ST3 | GCCTGGAATGGGTTGCADHTATTAGCACC CCCCGCGGGTCTDMTACCDACTATAGCGATAGCGTCAAGGG |
| H3 P6.1 | GCCGTCTATTATTGTACCCGTDVKDVKDVKTTTGMTTACTGGGGTCAAGGAACC |
| H3 P6.2 | GCCGTCTATTATTGTACCCGTDVKDVKDVKATGGMTTACTGGGGTCAAGGAACC |
| H3 P7.1 | GCCGTCTATTATTGTACCCGTDVKDVKDVKDVKTTTGMTTACTGGGGTCAAGGAACC |
| H3 P7.2 | GCCGTCTATTATTGTACCCGTDVKDVKDVKDVKATGGMTTACTGGGGTCAAGGAACC |
| H3 P8.1 | GCCGTCTATTATTGTACCCGTDVKDVKDVKDVKDVKTTTGMTTACTGGGGTCAAGGAACC |
| H3 P8.2 | GCCGTCTATTATTGTACCCGTDVKDVKDVKDVKDVKATGGMTTACTGGGGTCAAGGAACC |
| H3 P9.1 | GCCGTCTATTATTGTACCCGTDVKDVKDVKDVKDVKDVKTTTGMTTACTGGGGTCAAGGAACC |
| H3 P9.2 | GCCGTCTATTATTGTACCCGTDVKDVKDVKDVKDVKDVKATGGMTTACTGGGGTCAAGGAACC |
| H3 P10.1 | GCCGTCTATTATTGTACCCGTDVKDVKDVKDVKDVKDVKDVKTTTGMTTACTGGGGTCAAGGAACC |
| H3 P10.2 | GCCGTCTATTATTGTACCCGTDVKDVKDVKDVKDVKDVKDVKATGGMTTACTGGGGTCAAGGAACC |
| H3 P11.1 | GCCGTCTATTATTGTACCCGTDVKDVKDVKDVKDVKDVKDVKDVKTTTGMTTACTGGGGTCAAGGAACC |
| H3 P11.2 | GCCGTCTATTATTGTACCCGTDVKDVKDVKDVKDVKDVKDVKDVKATGGMTTACTGGGGTCAAGGAACC |
| H3 P12.1 | GCCGTCTATTATTGTACCCGTDVKDVKDVKDVKDVKDVKDVKDVKDVKTTTGMTTACTGGGGTCAAGGAACC |
| H3 P12.2 | GCCGTCTATTATTGTACCCGTDVKDVKDVKDVKDVKDVKDVKDVKDVKATGGMTTACTGGGGTCAAGGAACC |

To make the PS antibody libraries, two scFv templates were constructed, which included the pSAb and pSTab variable light chain linked to the variable heavy chain by a (Gly₄Ser)₃ linker, from pSAb and pSTAb and introduced two stop codons in the CDR H3. These plasmids were then used as templates for Kunkel mutagenesis. The light chain CDR L3 was diversified at positions 91-94 and 96 and the heavy chain CDR H2 was diversified at positions 50, 56, and 58 using degenerate codons designed to mimic the natural sequence diversity found at these positions (Table 8) (Sidhu, S. S. et al. Phage-displayed antibody libraries of synthetic heavy chain complementarity determining regions. J Mol Biol 338, 299-310 (2004) and Bostrom, J. et al. Variants of the antibody herceptin that interact with HER2 and VEGF at the antigen binding site. Science 323, 1610-1614 (2009)). CDR H3 was diversified using three to nine random amino acids (DVK) followed by three terminal residues (F/M, A/D, and Y) commonly observed in anti-peptide antibodies. For the mutagenesis reactions, L3 oligonucleotides (P1 and P2) were mixed at a 1:1 molar ratio, H2 oligonucleotides (1, 2, and 3) were mixed at a 0.1:1:2 ratio and H3 oligonucleotides (PX.1 and PX.2, where X=CDR length) were mixed at a 2:1 ratio. The resulting libraries were produced using Hyperphage (Rondot, S., Koch, J., Breitling, F. & Dubel, S. A helper phage to improve single-chain antibody presentation in phage display. Nat Biotechnol 19, 75-78 (2001)) to enhance recovery of rare binders and the final diversities of the pSAb and pSTAb libraries were 3.4×10¹⁰ and 2.7×10¹⁰, respectively.

TABLE 8
Amino acid diversity of phosphoscaffold libraries.

| Position | Amino acid diversity |
|---|---|
| L91 | Y, S, R, G, H, D |
| L92 | Y, N, S, D, T, A |
| L93 | N, T, G, D, A |
| L94 | S, T, Y, L, F, A, P, V, I, N, D, H |
| L96 | F, I, L, V |
| H50 | E, R, W, G, Y, V, I, A, N, S, D, F, T |
| H56 | Y, N, S, D, T, A |
| H58 | Y, D, N |
| H95-X* | Y, A, R, H, D, S, T, N, P, G, W, C, stop |
| H100C | F, M |
| H101 | D, A |

*H3 CDRs (H95-H102) were six to twelve amino acids in length.

### Phage Display Sections and ELISAs

All phage preparations and ELISAs were performed according to standard protocols (Sidhu, S. S. et al. Phage-displayed antibody libraries of synthetic heavy chain complementarity determining regions. J Mol Biol 338, 299-310 (2004) and Sidhu, S. S., Lowman, H. B., Cunningham, B. C. & Wells, J. A. Phage display for selection of novel binding peptides. Methods Enzymol 328, 333-363 (2000)). Briefly, 96-well Maxisorp plates were coated with 10 μg/mL NeutrAvidin overnight at 4° C. and subsequently blocked with 2% BSA for two hours at 20° C. Various concentrations of Fab-phage were mixed with a fixed concentration of biotinylated peptide and captured on the NeutrAvidin-coated wells for fifteen minutes. The bound phage were then detected using a horseradish peroxidase (HRP)-conjugated anti-M13 monoclonal (GE Healthcare). For phage competition ELISAs, plates were coated with 10 μg/mL NusA-KGNYVVTDH (the native target for the 1i8i Fab and a weak binder to pSAb, pSTAb, and pYAb), and blocked with 2% BSA. Sub-saturating levels of phage were then pre-bound to the various peptide antigens for two hours at 20° C. and then captured on the NusA-peptide coated plates for fifteen minutes. For scFv-Fc competition ELISAs, plates were coated with NeutrAvidin, blocked with 2% BSA, incubated with 100-200 nM biotinylated peptide, and finally blocked with 200 μM biotin. scFv-Fcs were then pre-bound to a dilution series of peptide antigen and processed as described for phage. Bound scFv-Fc were detected with HRP-conjugated Protein A (Pierce). Competition ELISA data was fit using a four-parameter logistic equation, with error shown by standard deviation of 2-3 replicates for each sample analyzed.

Selections with the H2-targeted libraries were performed using biotinylated phosphopeptide antigens captured with streptavidin-coated magnetic beads (Promega) (Table 5). In total, four rounds of the selection were performed with decreasing amounts of peptide antigen (500, 250, 100, and 10 nM) and individual phage clones were analyzed from the fourth round of selection. Selections with the pSAb and pSTAb libraries were identically performed except only three rounds were conducted.

### Example 2

### Characterization of Phospho-Specific (PS) Antibody Scaffolds

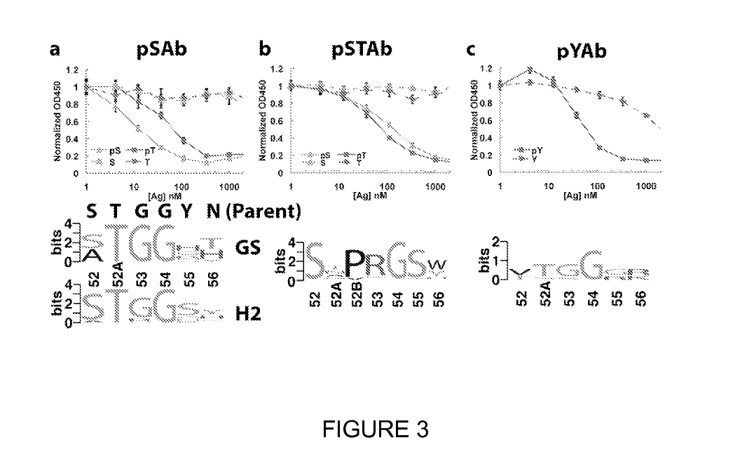

For each phosphopeptide antigen, single phage clones were isolated from each library and analyzed binding to the phosphopeptide by single-point ELISA (data not shown). For clones that gave ELISA signals >20-fold above background, the CDR H2 region was sequenced and sequences were constructed. Selections against the pSer and pThr peptides gave similar sequence logos and thus were combined into one logo. Analysis of the sequence logos from the H2- and GS-library selections against pSer/pThr highlighted the conservation of the key anchoring residue T52A_{H} and conformation residue G54_{H} in the loop, whereas more diversity was observed in the specificity residues (55_{H} and 56_{H}) (FIG. 3a). In the H2+1 libraries, a strong enrichment for a Pro-Arg insertion was observed in place of G53_{H} and complete conservation of G54_{H} (FIG. 3b). The G54_{H} residue occupies a region of the Ramachandran plot in which only glycine is allowed, thus suggesting that this glycine is critical for the conformation (Landry, R. C. et al. Antibody recognition of a conformational epitope in a peptide antigen: Fv-peptide complex of an antibody fragment specific for the mutant EGF receptor, EGFRvIII. J Mol Biol 308, 883-893 (2001); Hollingsworth, S. A. & Karplus, P. A. A fresh look at the Ramachandran plot and the occurrence of standard structures in proteins. Biomol Concepts 1, 271-283 (2010); and North, B., Lehmann, A. & Dunbrack, R. L., Jr. A new clustering of antibody CDR loop conformations. J Mol Biol 406, 228-256 (2011)). The pTyr Abs contained a different binding motif from the pSer/pThr clones consisting of a valine at 52_{H}, the Gly-Gly motif, and two consecutive basic residues, suggesting that the mode of pTyr recognition differs from that of pSer/pThr recognition (FIG. 3c).

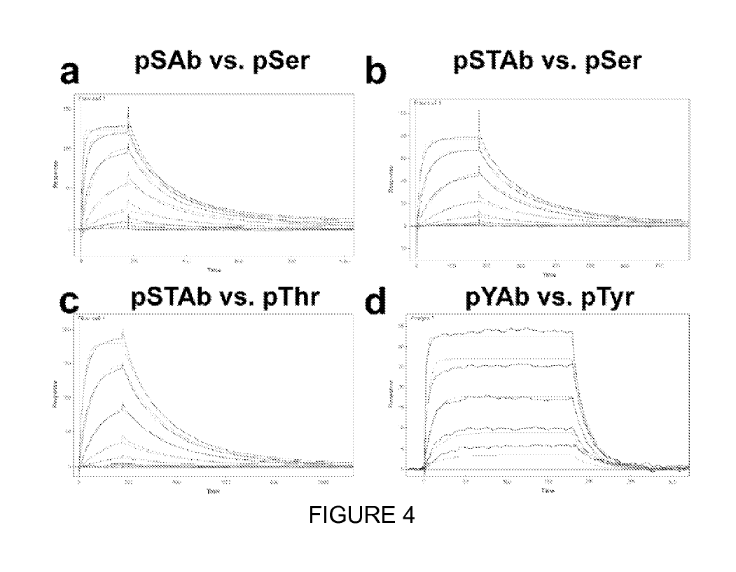

The phage clones by competition ELISA (as described above) were next analyzed to identify the best scaffold for each PTM target (pSer, pThr, or pTyr) (data not shown). From these experiments, a pSer-specific scaffold (pSAb with the loop sequence ATGGHT), a pSer/pThr-specific scaffold (pSTAb with sequence STPRGST), and a pTyr-specific scaffold (pYAb with sequence VTGGRK) were observed. To determine the phospho-selectivity of these scaffolds, binding to the phosphorylated and unphosphorylated peptides was analyzed. High selectivity for the phosphorylated peptide was observed in all cases (FIG. 3). For both the pSAb and pSTAb scaffolds, no binding to the unphosphorylated Ser or Thr peptides was observed up to 2 μM (FIGS. 3a and 3b). pSAb bound to pSer ˜10-fold tighter than pThr whereas pSTAb bound similarly to both pSer and pThr. pYAb bound >20-fold better to the pTyr peptide versus the unphosphorylated version (FIG. 3c). The specificities and binding affinities by surface plasmon resonance using purified Fabs and biotinylated peptides were confirmed (FIG. 4; Table 6).

### Biacore Analysis

Surface plasmon resonance data were measured on a Biacore model 4000 (Biacore, Uppsala, Sweden). All proteins were in TBS containing 0.1 mg/mL BSA and 0.01% Tween-20. A Biacore CM5 chip was coated with NeutrAvidin at 3000 RU and biotinylated antigens were captured at <100 RU. Serial dilutions of the Fabs were flowed over the immobilized antigens and 1:1 Langmuir binding models were used to calculate the kₒₙ, k_{off}, and K_{D} for each Fab:antigen pair.

### Example 3

### Structural Analysis of Phosphopeptide Recognition

To explore the mode of phosphoresidue recognition using X-ray crystallography, four Fab:peptide complexes (sSAb:pSer, pSTAb:pSer, pSTAb:pThr, and pYAb:pTyr) and the unbound pYAb Fab were expressed, purified, and crystallized. To express the Ab scaffolds, selected Fabs were amplified by PCR from the phage display vector, cloned into pJK4, and transformed into the C43 (DE3) bacterial strain for periplasmic protein expression. Protein expression was induced with 1 mM IPTG and the culture was grown overnight (18-20 hrs) at 30° C. A high level (up to ˜50%) of proteolyzed Fabs was initially observed after expression. Similar results were observed in other bacterial strains. Recombineering was used to knockout the genes that encode for the degP and prc proteases in the isogenic strain C43 (DE3) to generate the PRO (ΔdegP Δprc Δomp7) strain. Recombineering was performed according to standard protocols (Sharan, S. K., Thomason, L. C., Kuznetsov, S. G. & Court, D. L. Recombineering: a homologous recombination-based method of genetic engineering. Nat Protoc 4, 206-223 (2009)) to replace degP with a cassette encoding kanamycin resistance and prc with a cassette encoding tetracycline resistance. Additionally, a mutagenic oligonucleotide was used to introduce a W148R mutation in spr to correct the thermosensitive phenotype of prc knockout strains (Chen, C. et al. High-level accumulation of a recombinant antibody fragment in the periplasm of Escherichia coli requires a triple-mutant (degP prc spr) host strain. Biotechnol Bioeng 85, 463-474 (2004)) to generate the PRO+ strain. When Fabs were expressed in the C43 PRO+ strain, <5% of the Fab was proteolyzed (data not shown).

Expressed Fabs were purified from total cell lysates by Protein A chromatography, ion exchange chromatography, and a gel filtration chromatography step as previously described (Sidhu, S. S. et al. Phage-displayed antibody libraries of synthetic heavy chain complementarity determining regions. J Mol Biol 338, 299-310 (2004) and Bostrom, J. et al. Variants of the antibody herceptin that interact with HER2 and VEGF at the antigen binding site. Science 323, 1610-1614 (2009)). Fabs were stored at 4° C. for short-term analysis or flash frozen in 10% glycerol for storage at −80° C. Selected scFvs were PCR amplified and fused to a rabbit Fc domain (rFc) in a mammalian expression vector (pJK6). These constructs were transiently transfected into 293T cells and the resulting scFv-Fc proteins were purified from the media using Protein A chromatography. Nonphosphorylated versions of all peptides were fused to the C-terminus of NusA, which contained an N-terminal His₆ tag and biotin acceptor peptide and were co-expressed in BL21 (DE3) cells with BirA to enzymatically biotinylate each protein (pJK5). Recombinant proteins were purified on a His GraviTrap column (GE Healthcare, Piscataway, N.J.) followed by monomeric Avidin resin (Thermo Scientific, Rockford, Ill.) to a final purity of >95%. All biotinylated peptides were purchased from Elim Biopharmaceuticals (Hayward, Calif.) or Peptibody, Inc. (Charlotte, N.C.).

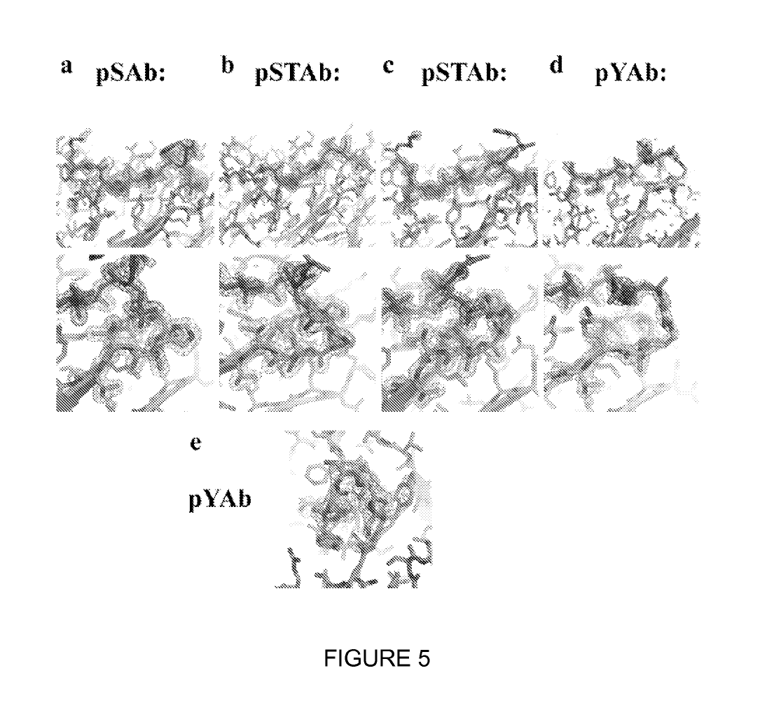

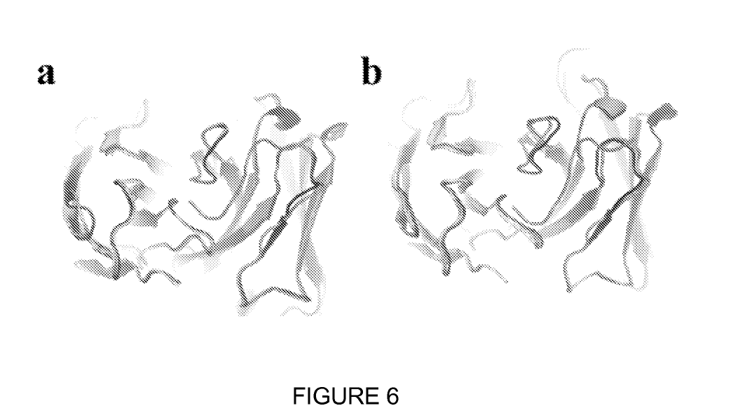

Four Fab:peptide complexes were successfully crystallized (pSAb:pSer, pSTAb:pSer, pSTAb:pThr, and pYAb:pTyr) as well as the unbound pYAb Fab (Table 9). Crystals for all four Fab:peptide complexes diffracted to better than 2 Å, whereas the unbound Fab diffracted to 2.63 Å (Table 9). Strong electron density for the bound peptide was observed in all pSer and pThr structures (FIG. 5). For the pYAb Fab, only one of the two Fab copies in the asymmetric unit was fully occupied by the peptide, likely due to the packing arrangement of the Fabs (FIG. 5). No changes in the positions of the CDRs were observed between the mouse 31 and humanized antibodies, which aligned with a ca RMSD of 0.78 Å. Furthermore, binding of the peptide to the antibody did not induce any major CDR movements (caRMSD of 1.3 Å) (FIG. 6).

TABLE 9
Crystallization and cryoprotection conditions for Fab complexes.

| | | Cryoprotectant | Temperature |
|---|---|---|---|
| Protein | Condition | solution | (° C.) |
| pSAb:pSer | 23% PEG1500, | Mother liquor | 4 |
| | 0.1M PCB | with 10% PEG200 | |
| | pH 6.8 | and 25% PEG1500 | |
| pSTAb:pSer | 22% PEG1500, | Mother liquor | 4 |
| | 0.1M PCB | with 10% PEG200 | |
| | pH 6.4 | and 25% PEG1500 | |
| pSTAb:pThr | 25% PEG1500, | Mother liquor | 4 |
| | 0.1M PCB | with 10% PEG200 | |
| | pH 6 | and 25% PEG1500 | |
| pYAb:pTyr | 20% PEG3350, | Mother liquor | 4 |
| | 0.2M KCl | with 10% PEG200 | |
| | | and 25% PEG3350 | |
| pYAb | 25% PEG1500, | Mother liquor | 18 |
| | 0.1M MMT | with 10% PEG200 | |
| | pH 4 | and 25% PEG1500 | |

TABLE 10
Data collection and refinement statistics (molecular replacement)

| | pSTAb:pThr | pSTAb:pSer | pSAb:pSer | pYAb:pTyr | pYAb |
|---|---|---|---|---|---|
| Data collection | | | | | |
| Space group | P 2₁ 2₁ 2₁ | P 2₁ 2₁ 2₁ | P 2₁ 2₁ 2₁ | P 3₂ 2 1 | P 3₂ 2 1 |
| Cell dimensions | | | | | |
| a, b, c (Å) | 43.81, 95.59, | 43.95, 95.89, | 43.5, 94.87, | 152.85, 152.85, | 152.26, 152.26, |
| | 119.82 | 119.92 | 120.58 | 85.29 | 83.55 |
| α, β, γ (°) | 90, 90, 90 | 90, 90, 90 | 90, 90, 90 | 90, 90, 120 | 90, 90, 120 |
| Resolution (Å) | 50-1.55 | 74.89-1.81 | 74.56-1.75 | 50-1.95 | 76.13-2.63 |
| | (1.604-1.55) | (1.875-1.81) | (1.813-1.75) | (2.02-1.95) | (2.724-2.63) |
| R_{sym} or R_{merge} | 0.065 (0.51) | 0.119 (0.67) | 0.113 (0.71) | 0.097 (0.67) | 0.115 (0.97) |
| I/σI | 15.22 (2.39) | 6.14 (1.90) | 7.22 (1.90) | 9.32 (2.11) | 11.61 (1.86) |
| Completeness (%) | 97.85 (86.93) | 99.70 (99.48) | 99.49 (99.33) | 99.92 (99.81) | 99.49 (95.97) |
| Redundancy | 5.6 (2.9) | 3.8 (3.8) | 3.9 (3.9) | 4.1 (4.1) | 7.8 (5.4) |
| Refinement | | | | | |
| Resolution (Å) | 50-1.55 | 74.89-1.81 | 74.56-1.75 | 50-1.95 | 76.13-2.63 |
| No. reflections | 72503 (3659) | 46969 (2418) | 50977 (2622) | 83503 (4166) | 33257 (1719) |
| R_{work}/R_{free} (%) | 15.0/17.2 | 16.1/20.2 | 15.4/19.9 | 16.3/20.2 | 18.8/23.6 |
| No. atoms | | | | | |
| Protein | 3607 | 3461 | 3470 | 6764 | 6543 |
| Ligand | 5 | 5 | 5 | 26 | 52 |
| Water | 623 | 615 | 675 | 977 | 56 |
| Wilson B-value (Å) | 13.33 | 18.36 | 15.42 | 23.38 | 59.75 |
| B-factors | | | | | |
| Protein | 18.4 | 23.2 | 19 | 32 | 82.6 |
| Water | 31.0 | 34.0 | 30.7 | 38.9 | 61.7 |
| R.m.s. deviations | | | | | |
| Bond lengths (Å) | 0.009 | 0.003 | 0.01 | 0.009 | 0.007 |
| Bond angles (°) | 1.31 | 0.88 | 1.32 | 1.2 | 0.86 |
| Ramachandran statistics (%) | | | | | |
| Favored | 98 | 98 | 98 | 98 | 97 |
| Outliers | 0.2³ | 0.2³ | 0.2⁴ | 0 | 0.2⁴ |

¹Values in parentheses are for highest-resolution shell.
²Data was collected from a single crystal for each structure.
³Outlier residue (Pro52B_{H}) is the same in both structure with excellent density.
⁴Outlier residue (Pro149_{H}) is the same in both structure with excellent density in the high resolution structure.

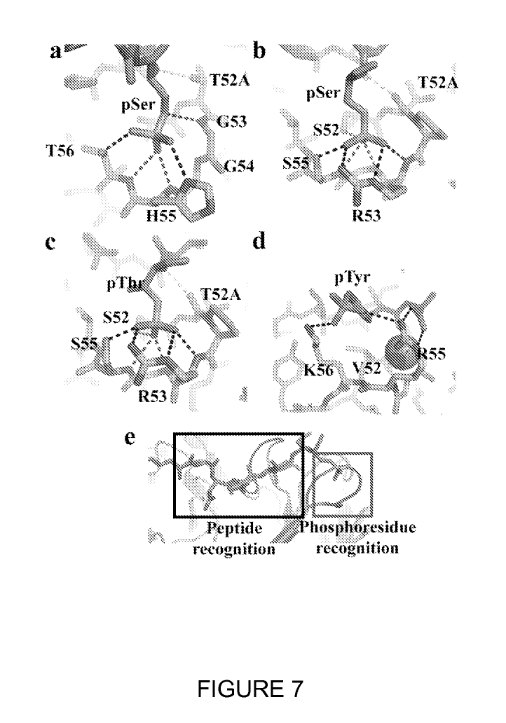

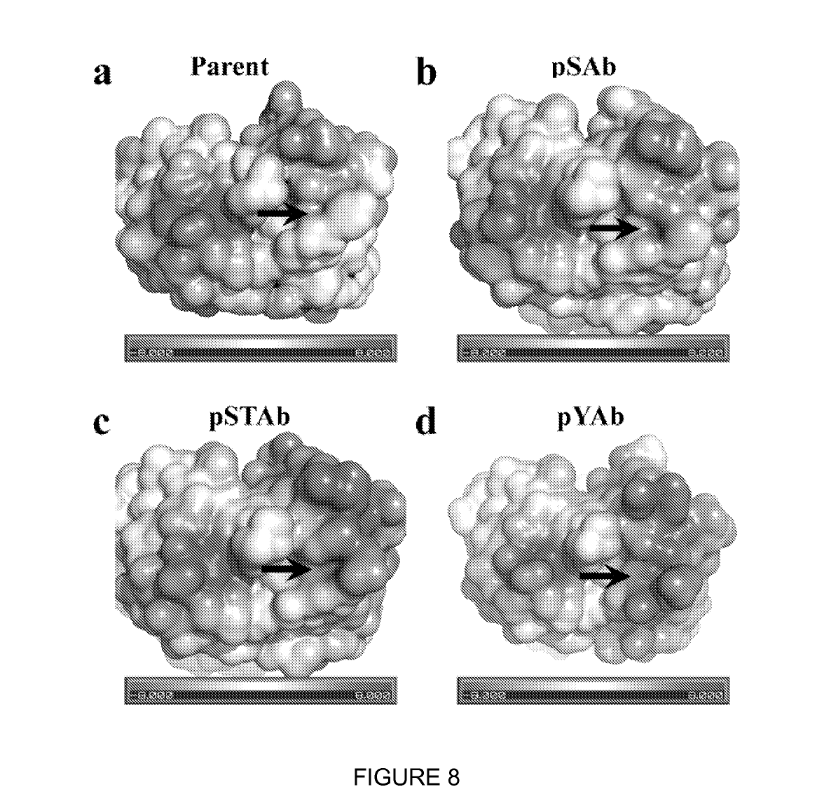

The X-ray structures of the parent peptide:Fab complexes illustrate how CDR H2 specifically recognizes each phosphoresidue (FIG. 7). For all three scaffolds, mutations found in the parent H2 loop make the main-chain more accessible and create a large electropositive hot spot (indicated by arrow in FIG. 8). The phosphoresidue side chain is almost fully engulfed by the Ab in pSAb (80% buried) and pSTAb (92% buried) and anchored by multiple hydrogen bonds to side chains and main-chain amides (five and seven bonds for pSAb and pSTAb, respectively), confirming that the hot spot was successfully optimized in the CDR (FIG. 7a, b, c, and Table 11). In pSAb, the pSer residue makes key contacts with specificity residues G53_{H}, R55_{H}, and T56_{H}, whereas in pSTAb, the pSer and pThr residues make key contacts with R53_{H}, G54_{H}, and S55_{H}. In pSTAb, the insertion of P52B_{H} allows the T52A_{H} anchor to flip out and still contribute a hydrogen bond from the main-chain carbonyl. In contrast, pYAb does not utilize the original designed loop conformation to bind pTyr (FIG. 7d). A key ionic interaction between H56K and the phosphate of pTyr and a hydrophobic interaction between V52_{H} and the phenyl ring contribute to the recognition mode. The H2 nest pocket is occupied by a water molecule that is additionally stabilized by the free C-terminus of the peptide, indicating that pYAb may bind differently to the pTyr residue in longer peptides without this neighboring free carboxylate (FIG. 7d). For all phosphopeptides, the recognition is achieved through two sectors: the phosphoresidue-binding pocket and neighboring peptide sequence “reader” region, which consists primarily of CDRs L3 and H3 (FIG. 7e). Combined, the in vitro characterization and X-ray crystal structures confirmed that novel Ab scaffolds were designed that utilize pSer, pThr, or pTyr as hot-spot residues.

TABLE 11
Contact analysis between phosphopeptides
and various binding domains

| | Percent of | Number of | |
|---|---|---|---|
| | phospho- | hydrogen bonds | |
| | residue | between phospho- | |
| | buried | residue | |
| Domain | in complex | and domain | Reference |
| 14-3-3 (pSer) | 89.3 | 6 | Wilker et al. (74) |
| WW (pSer) | 79.4 | 5 | Verdecia et al. (75) |
| BRCT (pSer) | 69.2 | 3 | Clapperton et al. (76) |
| FHA (pThr) | 62.2 | 4 | Byeon et al. (78) |
| WD40 (pThr) | 66.1 | 5 | Orlicky et al. (77) |
| SH2 (pTry) | 91.6 | 4 | Mulhern et al. (79) |
| PTB (pTry) | 74.3 | 4 | Zhou et al. (80) |
| Chicken scFv | 77.7 | 4 | Tu et al. (73) |
| (pThr) | | | |
| pSAb (pSer) | 80 | 5 | Herein |
| pSTAb (pSer) | 92 | 7 | Herein |
| pSTAb (pThr) | 92.7 | 7 | Herein |
| pYAb (pTyr) | 38.3 | 3 | Herein |

### Crystallization of Peptide:Fab Complexes

Fabs were expressed as described herein and concentrated to 10-15 mg/mL in 10 mM Tris pH7.5, 50 mM NaCl. Complexes of the Fab with the corresponding peptide were formed at a 1:2 molar ratio of Fab:peptide. Crystals were grown in hanging drop format by mixing 100 nL of protein solution and 100 nL crystallization solution using a Mosqutio nanoliter pipetting system (TTP Labtech). Crystals formed within one to two weeks at either 18° C. or 4° C. A microseeding strategy was generated with a seed stock generated from finely ground pSTAb:pThr crystals in 50 μL cryoprotectant solution (Luft, J. R. & DeTitta, G. T. A method to produce microseed stock for use in the crystallization of biological macromolecules. Acta Crystallogr D Biol Crystallogr 55, 988-993 (1999)). Crystals for the pSAb:pSer and pSTAb:pSer complexes were generated by hanging drop vapor diffusion with 300 nL drops consisting of 150 nL protein solution, 120 nL reservoir solution, and 30 nL 1:100 dilution of seed stock. All crystals were soaked in cryoprotectant solution and flash frozen in liquid nitrogen. Crystallization conditions and cryoprotectant solutions are listed in Table 9.

Diffraction data were collected using the Advanced Light Source beam line 8.3.1 at the Lawrence Berkeley National Laboratory (Berkeley, Calif.) with a wavelength of 1.1 Å. The data were indexed, integrated, and scaled using ELVES (Holton, J. & Alber, T. Automated protein crystal structure determination using ELVES. Proc Natl Acad Sci USA 101, 1537-1542 (2004)) and HKL2000 (Otwinowski, Z. & Minor, W. Processing of X-ray diffraction data collected in oscillation mode. Method Enzymol 276, 307-326 (1997)). The structure of the pSTAb:pThr complex was solved by molecular replacement using Phenix (Otwinowski, Z. & Minor, W. Processing of X-ray diffraction data collected in oscillation mode. Method Enzymol 276, 307-326 (1997)). The initial search model consisted of the variable heavy domain from 3n9g and the variable light domain, constant heavy domain, and constant light domain from 2gcy55. The pSTAb Fab structure was used as the search model for all other structures. Iterative rounds of model building and refinement were carried out with Phenix and Coot (Emsley, P. & Cowtan, K. Coot: model-building tools for molecular graphics. Acta Crystallogr D Biol Crystallogr 60, 2126-2132 (2004)). For isomorphous crystals, the same refinement test sets for calculating Rfree were used. Simulated annealing composite omit maps calculated using Phenix were used to remove model bias. After two rounds of refinement, peptides were built into each model using Coot. Riding hydrogens as implemented in Phenix were used in the final stages of refinement for the pSAb:pSer, pSTAb:pSer, and pSTAb:pThr complexes. Final refinement statistics can be found in Table 10. The final coordinates were validated using MolProbity (Chen, V. B. et al. MolProbity: all-atom structure validation for macromolecular crystallography. Acta Crystallogr D Biol Crystallogr 66, 12-21 (2010)). The final Ramachandran statistics % Favored:% Outlier) were 98:0.2, 98:0.2, 98:0.2, 98:0, and 97:0.2 for pSAb:pSer, pSTAb:pSer, pSTAb:pThr, pYAb:pTyr, and pYAb, respectively. MacPyMol (DeLano Scientific) was used to generate structure figures. Electrostatic surfaces were calculated using APBS (Baker, N. A., Sept, D., Joseph, S., Holst, M. J. & McCammon, J. A. Electrostatics of nanosystems: application to microtubules and the ribosome. Proc Natl Acad Sci USA 98, 10037-10041 (2001)) and buried surface areas were calculated using CCP4 (Winn, M. D. et al. Overview of the CCP4 suite and current developments. Acta Crystallogr D Biol Crystallogr 67, 235-242 (2011)).

### Example 4

### Generation of Novel Phospho-Specific Antibodies Using pSer and pSer/pThr Scaffolds

Because every member of the initial library contains a phospho-hot spot, each Ab is should have a weak initial affinity for the phosphorylated antigen and this anchor should dramatically enhance the selection of new antibodies. As a proof of principle, pSer- and pThr-containing antigens were targeted because reagents capable of detecting these modifications are lacking in the art. The pSAb and pSTAb Fab scaffolds were converted into single chain fragment variable (scFv) scaffolds to improve the display level on phage and ultimately, improve the selection of Abs from the library. The surface-exposed positions were diversified in CDR H2 (50_{H}, 56_{H}, and 58_{H}) outside of the phosphate-hot spot and CDR L3 (91_{L}-94_{L}, 96_{L}) using codons designed to mimic natural Ab sequence diversity (Sidhu, S. S. et al. Phage-displayed antibody libraries of synthetic heavy chain complementarity determining regions. J Mol Biol 338, 299-310 (2004)) (Table 8). CDR H3 was diversified with CDR lengths ranging from six to twelve amino acids using a degenerate codon designed to explore maximal chemical diversity, while still allowing efficient sampling of the sequence space in the library. The pSAb and pSTAb libraries had diversities of 3.4×10¹⁰ and 2.7×10¹⁰, respectively.

A set of ten biologically relevant pSer- or pThr-containing epitopes were chosen as target antigens (Table 12 and Table 5). Three rounds of selection were performed and single phage clones were analyzed from the third round of selection by single-point ELISA. For seven targets, at least one scFv was isolated that bound only to the phosphorylated and not the unphosphorylated antigen (Table 12 and FIG. 9a). To demonstrate that the isolated clones are specific to each phosphorylated peptide, a panel of ELISAs (as described above) was performed to assay binding of each scFv to each of the ten phosphorylated peptides (FIGS. 9b and 9c). The data demonstrated the exquisite target selectivity of most of the scFv clones, indicating the absence of promiscuous pSer-/pThr peptide binding scFvs. These scFvs efficiently expressed in both bacteria (2-10 mg/L) and mammalian 293T cells (as Fc fusions with yields of 0.5-5 μg/mL) (data not shown). Additionally, the scFv-Fc fusions exhibited affinities ranging from 42 to 2430 nM (Table 12), which matches or exceeds previous reports of PS antibody affinities (Feldhaus, M. J. et al. Flow-cytometric isolation of human antibodies from a nonimmune Saccharomyces cerevisiae surface display library. Nat Biotechnol 21, 163-170 (2003) and Shih, H. H. et al. An ultra-specific avian antibody to phosphorylated tau reveals a unique mechanism for phosphoepitope recognition. J Biol Chem (2012)). These library selections validate the strategy of using a designed PS scaffold to rapidly generate novel PS antibodies.

TABLE 12
Summary of scFv hits versus new phosphopeptide targets.

| | | | Number of | | |
|---|---|---|---|---|---|
| | | Number of | phospho- | | |
| Pep- | | unique | specific | | |
| tide | Target | scFvs | scFvs^{a} | K_{D} (nM)^{b} | |
| P1 | Caspase 3 | 5 | 0 | n.d. | |
| | (S12) | | | | |
| P2 | RIPK3 (S227) | 6 | 2 | 102 ± 15 | (P2.A11) |
| P3 | RIPK3 (S199) | 23 | 17 | 250 ± 13 | (P3.28) |
| P4 | Smad2 (T8) | 3 | 2 | 78 ± 14 | (P4.B9) |
| P5 | CREB (S133) | 4 | 4 | 151 ± 8 | (P5.G10) |
| P6 | HtrA2 (S212) | 21 | 21 | 2430 ± 150 | (P6.C12) |
| P7 | Akt1 (T308) | 0 | 0 | n.d. | |
| P8 | Akt1 (S473) | 1 | 1 | >5000^{c} | (P8.H9) |
| P9 | PKC theta | 1 | 0 | n.d. | |
| | (S695) | | | | |
| P10 | Sgk1 (S422) | 4 | 4 | 42.2 ± 2.8 | (P10.D6) |

^{a}scFv clones that exhibited >5-fold higher ELISA signal against phosphorylated peptide compared to unphosphorylated peptide (FIG. 9).
^{b}As determined by competition ELISA with scFv-Fc protein (n = 2-3). Clone ID is shown in parentheses.
^{c}Only partial competition was observed at the concentrations of peptide used.

### DISCUSSION

Described herein is a novel and renewable antibody generation method that entails the design of a motif-specific (e.g. pSer, pThr, or pTyr) antibody scaffold followed by structure-informed mutagenesis of the scaffold to generate monoclonal motif-specific antibodies against a panel of phosphopeptide antigens. The high success rate (70%), which does not employ counter selections against the unphosphorylated epitope demonstrates how the motif-specific hot spot greatly improves the selection process as even past Ab libraries generated from immunized animals required stringent counter selections to enrich for PS antibodies (Shih, H. H. et al. An ultra-specific avian antibody to phosphorylated tau reveals a unique mechanism for phosphoepitope recognition. J Biol Chem (2012) and Vielemeyer, O. et al. Direct selection of monoclonal phosphospecific antibodies without prior phosphoamino acid mapping. J Biol Chem 284, 20791-20795 (2009)). Structural validation of the hot spot prior to library generation allowed confirmation of the mode of recognition and to identify the key antibody residues involved in the recognition (FIG. 7). In the case of pSAb and pSTAb, the hot spot contains a hallmark α_{L} glycine at 54_{H} that contributes to the main-chain conformation of CDR H2. There is a remarkably high frequency of occurrence for this H2 conformation in Abs (˜12% of all H2 conformations (North, B., Lehmann, A. & Dunbrack, R. L., Jr. A new clustering of antibody CDR loop conformations. J Mol Biol 406, 228-256 (2011))). Moreover, eight Ab structures reveal an aspartate or glutamate bound to the same H2 hot spot, and three Ab structures have a sulfate ion bound. A recent structure of a chicken scFv, which was generated from an immunized phage display library with a phosphopeptide antigen, was reported that utilized a similar H2 conformation to bind pThr (Shih, H. H. et al. An ultra-specific avian antibody to phosphorylated tau reveals a unique mechanism for phosphoepitope recognition. J Biol Chem (2012)). Together these data suggest there may be a germline-encoded anion-hot spot capable of binding phosphate or sulfate groups (FIG. 2). The conservation of this site is intriguing because previous work on Abs that bind phospholipids suggested a “phosphate-binding subsite” that conferred recognition of only the phosphorylated or sulfated forms of multiple lipids and haptens (Alving, C. R. Antibodies to liposomes, phospholipids and phosphate esters. Chem Phys Lipids 40, 303-314 (1986)). Furthermore, anion-hot spot-containing Abs may provide a protective role in the recognition of phosphorylated or sulfated antigens, such as lipid A in Gram-negative bacteria (Alving, C. R. Antibodies to liposomes, phospholipids and phosphate esters. Chem Phys Lipids 40, 303-314 (1986).), or conversely, a more sinister role in autoimmune diseases, such as antiphospolipid syndrome (Levine, J. S., Branch, D. W. & Rauch, J. The antiphospholipid syndrome. N Engl J Med 346, 752-763 (2002)).

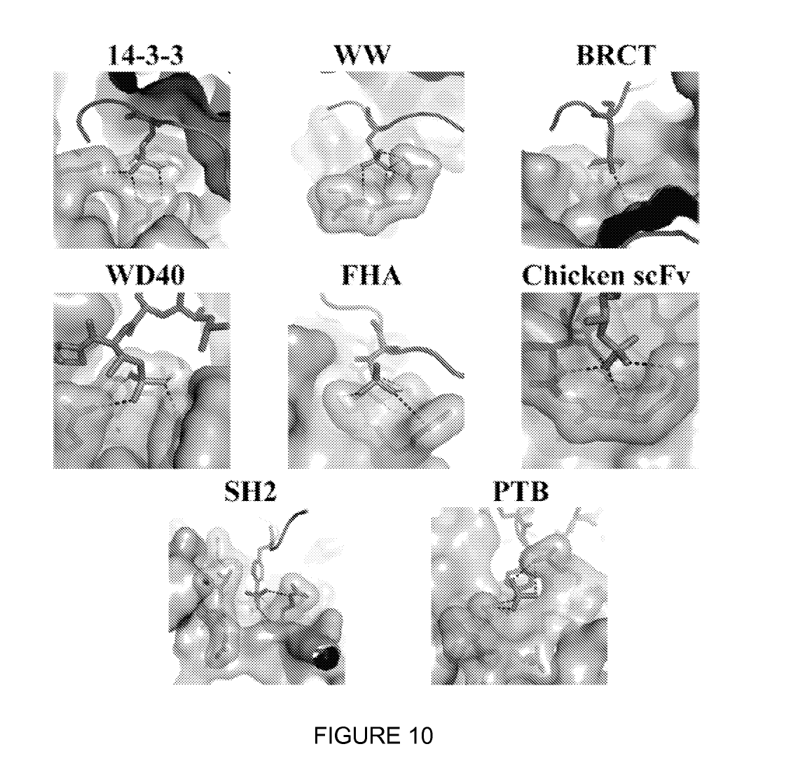

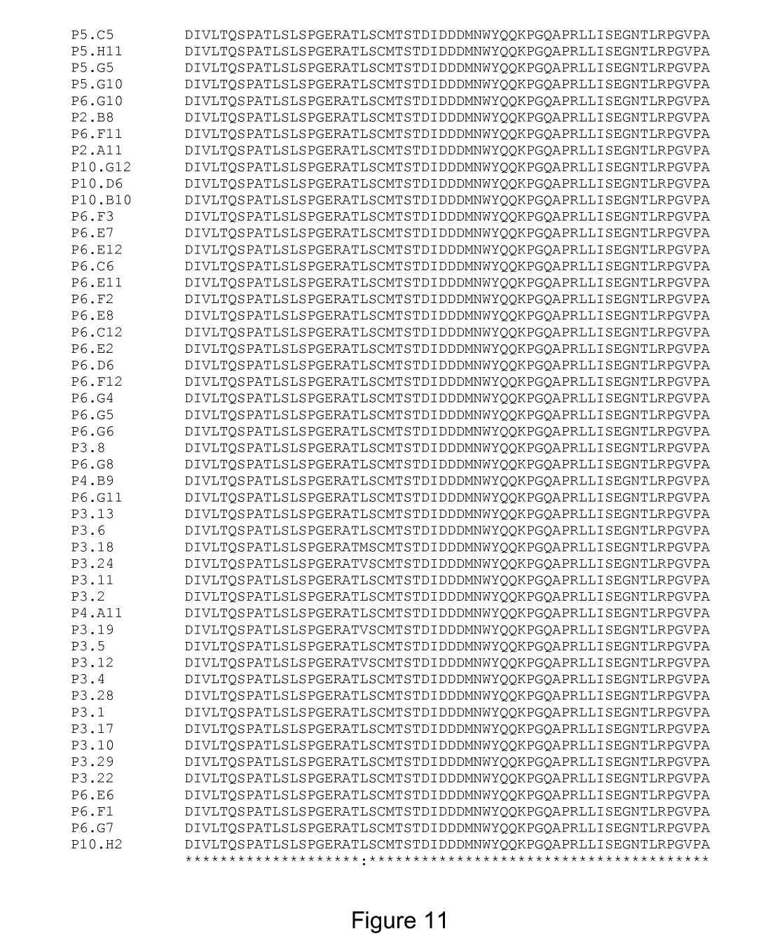

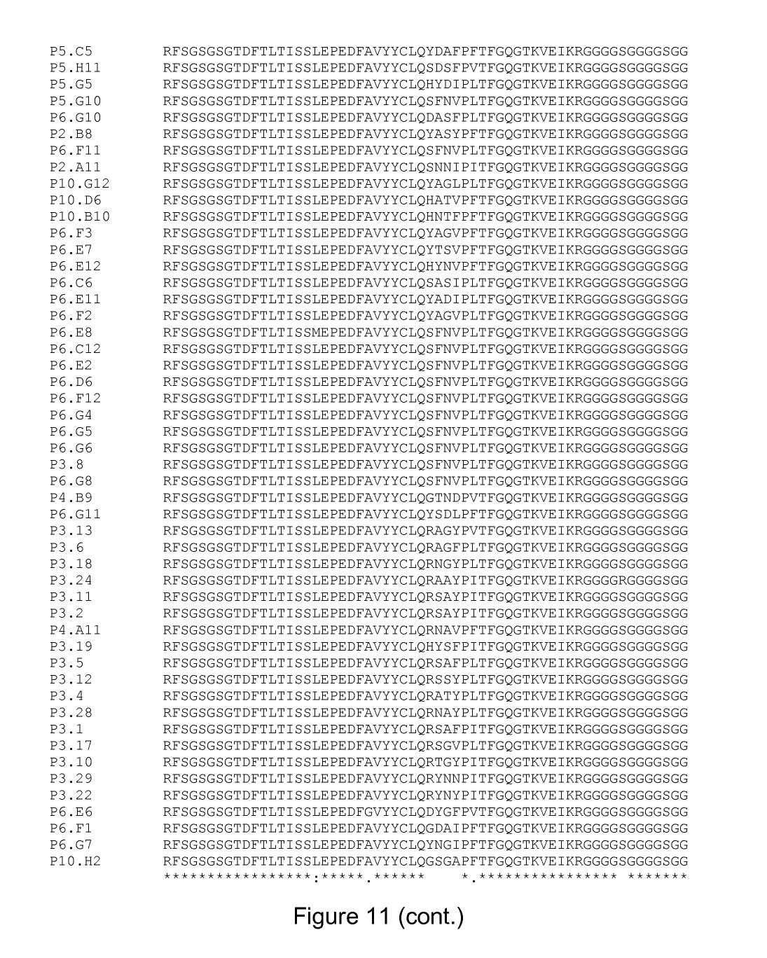

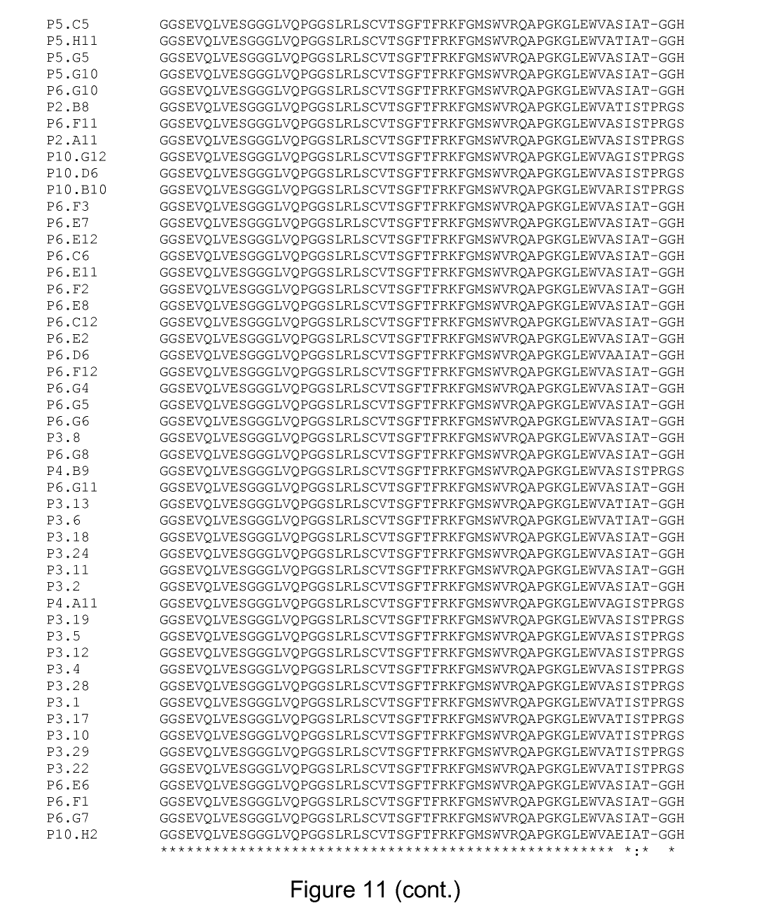

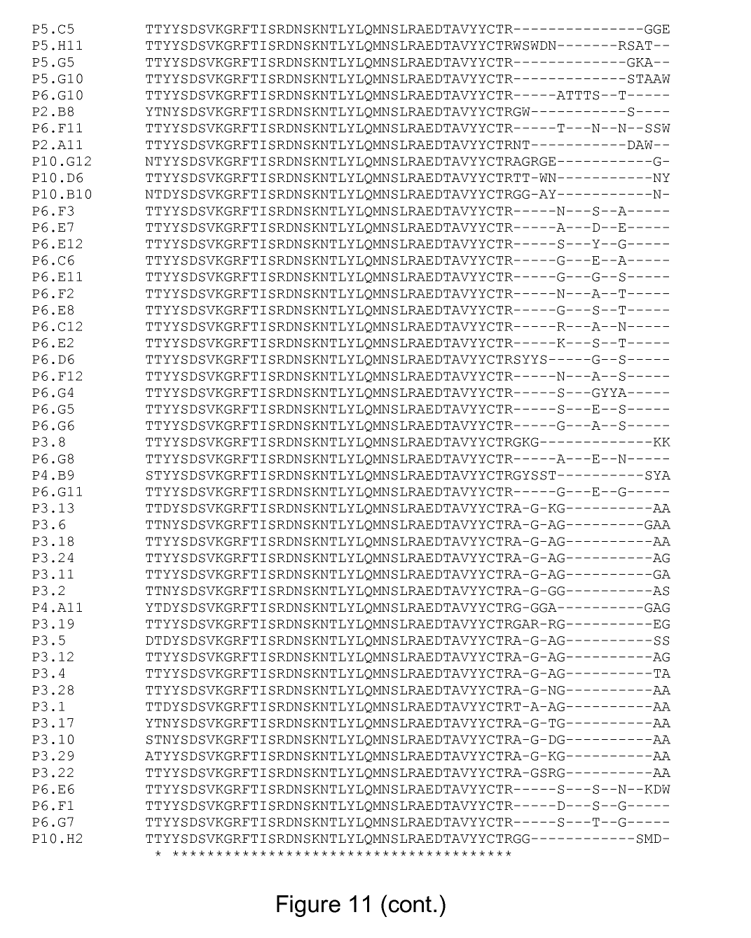

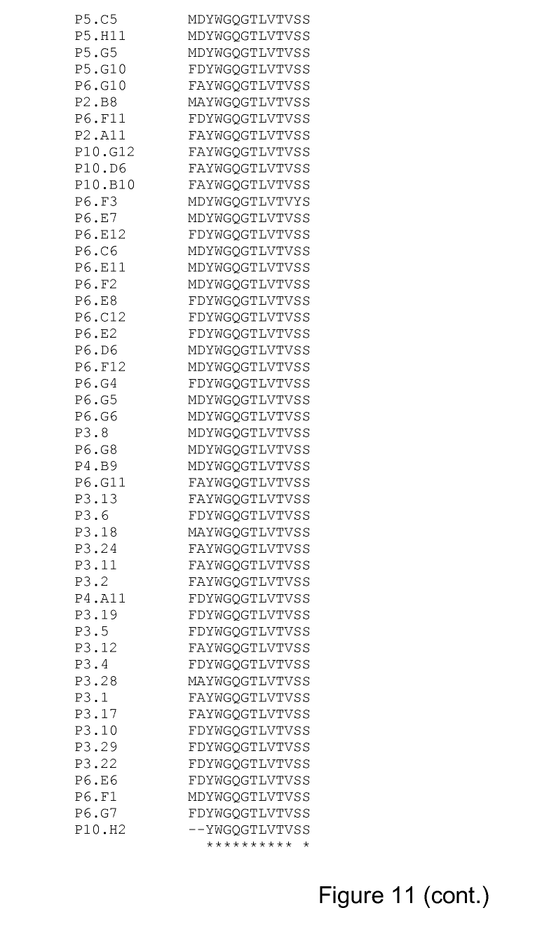

The main chain dominated mode of pSer/pThr recognition is completely different from most endogenous pSer/pThr-binding domains such as SH2, 14-3-3, and FHA, that predominantly utilize side chains to bind the phospho-residue (Yaffe, M. B. & Smerdon, S. J. PhosphoSerine/threonine binding domains: you can't pSERious? Structure 9, R33-38 (2001)) (FIG. 7 and FIG. 10). Only the WW domain sometimes utilizes two main-chain amides to bind a phosphate. In fact, the pSer/pThr scaffolds described herein bind more efficiently to the phosphoresidue than naturally occurring domains by burying a larger surface area and contributing more hydrogen bonds (Table 11). Others have recently suggested that these endogenous phosphoresidue-binding and other PTM-binding domains have evolved to bind shorter epitopes with moderate affinities to support the dynamic nature of signal transduction pathways, which potentially limits the range of epitopes they can bind (Yaffe, M. B. & Smerdon, S. J. PhosphoSerine/threonine binding domains: you can't pSERious? Structure 9, R33-38 (2001); Kaneko, T., Joshi, R., Feller, S. M. & Li, S. S. Phosphotyrosine recognition domains: the typical, the atypical and the versatile. Cell Commun Signal 10, 32 (2012); and Seet, B. T., Dikic, I., Zhou, M. M. & Pawson, T. Reading protein modifications with interaction domains. Nat Rev Mol Cell Biol 7, 473-483 (2006)). Additionally, the designed PS hot spots described herein function independently of other CDRs as those could be diversified to target highly diverse phosphopeptides (FIG. 9 and Table 5). Because many other PTM-binding motifs exist in nature, such motifs can be similarly designed into Abs to generate high-affinity monoclonal reagents capable of detecting other PTMs beyond phosphorylation.

pYAb utilizes a completely different motif to recognize pTyr (FIG. 7d). The salt bridge between the phosphate and H56 Lys is more akin to how 14-3-3 and PTB domains bind phosphorylated residues. Thus, optimization of a single CDR loop generated two different phosphate-recognition modes, both of which are found in natural protein domains (Kaneko, T., Joshi, R., Feller, S. M. & Li, S. S. Phosphotyrosine recognition domains: the typical, the atypical and the versatile. Cell Commun Signal 10, 32 (2012) and Hirsch, A. K., Fischer, F. R. & Diederich, F. Phosphate recognition in structural biology. Angew Chem Int Ed Engl 46, 338-352 (2007)), providing an example of convergent evolution among multiple protein folds. Furthermore, highly specific recognition of pTyr was obtained despite not burying most of the pTyr phenyl ring (FIG. 3c and FIG. 7d).

In stark contrast to traditional monoclonal or polyclonal PS Abs, the PS Abs described herein utilize a single framework that permits high-level bacterial expression (>3 mg/L) and mammalian expression (˜0.5-5 μg/mL media) in a renewable format. The use of a single framework greatly simplifies mutagenesis protocols (e.g. affinity maturation), sequence-function analysis, and conversion to other antibody formats (e.g. IgG) (Sidhu, S. S. et al. Phage-displayed antibody libraries of synthetic heavy chain complementarity determining regions. J Mol Biol 338, 299-310 (2004)). Additionally, the recombinant PS antibodies described herein can be genetically fused to a variety of molecules, thus permitting the rapid generation of detection reagents (e.g. Fc fusions) or intracellular probes (e.g. substance P fusions) (Rizk, S. S. et al. An engineered substance P variant for receptor-mediated delivery of synthetic antibodies into tumor cells. Proc Natl Acad Sci USA 106, 11011-11015 (2009)).

The ability to rapidly generate recombinant, monoclonal PS antibodies provides several future applications. One emerging application is the use of PTM-specific antibodies to immunoenrich biological samples for subsequent mass spectrometry analysis. For example, several pan-specific pTyr Abs, that recognize pTyr peptides with limited specificity for the neighboring residues, have revolutionized the study of pTyr signaling by permitting efficient enrichment of this low abundant phosphospecies (<0.05%) (Blagoev, B., Ong, S. E., Kratchmarova, I. & Mann, M. Temporal analysis of phosphotyrosine-dependent signaling networks by quantitative proteomics. Nat Biotechnol 22, 1139-1145 (2004) and Nita-Lazar, A., Saito-Benz, H. & White, F. M. Quantitative phosphoproteomics by mass spectrometry: past, present, and future. Proteomics 8, 4433-4443 (2008)). However, all of these antibodies have inherent sequence biases outside of the pTyr motif and thus, numerous pTyr sites are likely missed (Nita-Lazar, A., Saito-Benz, H. & White, F. M. Quantitative bhosphoproteomics by mass met: ast present, and future. Proteomics 8, 4433-4443 (2008) and Matsuoka, S. et al. ATM and ATR substrate analysis reveals extensive protein networks responsive to DNA damage. Science 316, 1160-1166 (2007)). Ideally, one would perform global identification of pTyr sites using an immunoenrichment reagent capable of isolating ˜XpYX˜, where X is any amino acid. Alternatively, one could focus on a subset of pTyr sites involved a particular signaling pathway (e.g. ˜(D/E)pY(I/L/V)˜ for EGFR kinase substrates) using a more tailored Ab reagent. In either case, one could develop a renewable and reproducible mixture of monoclonal PS Abs that enriches a degenerate phosphorylation motif, such as a consensus substrate motif for a kinase.

The Ab mixtures described herein are advantageous over traditional affinity-purified polyclonal Abs, which are not renewable and vary from batch to batch. For example, a recent study highlights the ability of a well-defined Ab mixture generated from two or three commercially available PS Abs against known ATM and ATR kinase substrates to serve as immunoaffinity reagents to identify >900 phosphorylation sites induced by DNA damage (Matsuoka, S. et al. ATM and ATR substrate analysis reveals extensive protein networks responsive to DNA damage. Science 316, 1160-1166 (2007)). The scFv clones P3.4 and P3.8 described herein represent the first components of a degenerate pSer-specific antibody pool that cross-reacts with multiple phosphorylated peptides (FIG. 9c). Sequential selections of the libraries against several phosphopeptides that share a common motif beyond the phosphoresidue can generate additional antibodies with tailored degenerate specificities.

The use of global-scale phosphoproteomics experiments to first identify potential phosphorylated biomarkers (Wang, Y. et al. Phosphorylated alpha-synuclein in Parkinson's disease. Sci Trans' Med 4, 121ra120 (2012) and Hampel, H. et al. Measurement of phosphorylated tau epitopes in the differential diagnosis of Alzheimer disease: a comparative cerebrospinal fluid study. Arch Gen Psychiatry 61, 95-102 (2004)) followed by rapid in vitro generation of monoclonal, PS Abs is a powerful diagnostic platform and potential therapeutic strategy (Tagliabracci, V. S. et al. Secreted kinase phosphorylates extracellular proteins that regulate biomineralization. Science 336, 1150-1153 (2012)) in the treatment of human disease. Additionally, the bacteriophage-derived PS Ab platform, which can be automated, rapidly generates Abs within two weeks as opposed to the several months required for hybridoma methods. Finally, the motif-specific scaffold method described herein can be generalize to the targeting of virtually any antigen with a defined motif.

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The examples set forth above are provided to give those of ordinary skill in the art a complete disclosure and description of how to make and use the embodiments of the compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated herein by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the disclosure is to be limited only by the terms of the appended claims, along with the full scope of equivalents to which the claims are entitled.

Listing of Antibody Sequences

>P2.A11
DIVLTQSPATLSLSPGERATLSCMTSTDIDDDMNWYQQKPGQAPRLLISEGNTLRPGVPARFSGSGSGTDFTLTIS
SLEPEDFAVYYCLQSNNIPITFGQGTKVEIKRGGGGSGGGGSGGGGSEVQLVESGGGLVQPGGSLRLSCVTSGFTF
RKFGMSWVRQAPGKGLEWVASISTPRGSTTYYSDSVKGRFTISRDNSKNTLYLQMNSLRAEDTAVYYCTRNT-----
DAW--FAYWGQGTLVTVSS
>P2.B8
DIVLTQSPATLSLSPGERATLSCMTSTDIDDDMNWYQQKPGQAPRLLISEGNTLRPGVPARFSGSGSGTDFTLTIS
SLEPEDFAVYYCLQYASYPFTFGQGTKVEIKRGGGGSGGGGSGGGGSEVQLVESGGGLVQPGGSLRLSCVTSGFTF
RKFGMSWVRQAPGKGLEWVATISTPRGSYTNYSDSVKGRFTISRDNSKNTLYLQMNSLRAEDTAVYYCTRGW-----
S----MAYWGQGTLVTVSS
>P3.29
DIVLTQSPATLSLSPGERATLSCMTSTDIDDDMNWYQQKPGQAPRLLISEGNTLRPGVPARFSGSGSGTDFTLTISS
LEPEDFAVYYCLQRYNNPITFGQGTKVEIKRGGGGSGGGGSGGGGSEVQLVESGGGLVQPGGSLRLSCVTSGFTFRK
FGMSWVRQAPGKGLEWVATISTPRGSATYYSDSVKGRFTISRDNSKNTLYLQMNSLRAEDTAVYYCTRA-G-KG-
AAFDYWGQGTLVTVSS
>P3.24
DIVLTQSPATLSLSPGERATVSCMTSTDIDDDMNWYQQKPGQAPRLLISEGNTLRPGVPARFSGSGSGTDFTLTISS
LEPEDFAVYYCLQRAAYPITFGQGTKVEIKRGGGGRGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
AGFAYWGQGTLVTVSS
>P3.22
DIVLTQSPATLSLSPGERATLSCMTSTDIDDDMNWYQQKPGQAPRLLISEGNTLRPGVPARFSGSGSGTDFTLTISS
LEPEDFAVYYCLQRYNYPITFGQGTKVEIKRGGGGSGGGGSGGGGSEVQLVESGGGLVQPGGSLRLSCVTSGFTFRK
FGMSWVRQAPGKGLEWVATISTPRGSTTYYSDSVKGRFTISRDNSKNTLYLQMNSLRAEDTAVYYCTRA-GSRG-
AAFDYWGQGTLVTVSS
>P3.18
DIVLTQSPATLSLSPGERATMSCMTSTDIDDDMNWYQQKPGQAPRLLISEGNTLRPGVPARFSGSGSGTDFTLTISS
LEPEDFAVYYCLQRNGYPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
AAMAYWGQGTLVTVSS
>P3.17
DIVLTQSPATLSLSPGERATLSCMTSTDIDDDMNWYQQKPGQAPRLLISEGNTLRPGVPARFSGSGSGTDFTLTISS
LEPEDFAVYYCLQRSGVPLTFGQGTKVEIKRGGGGSGGGGSGGGGSEVQLVESGGGLVQPGGSLRLSCVTSGFTFRK
FGMSWVRQAPGKGLEWVATISTPRGSYTNYSDSVKGRFTISRDNSKNTLYLQMNSLRAEDTAVYYCTRA-G-TG-
AAFAYWGQGTLVTVSS
>P3.13
DIVLTQSPATLSLSPGERATLSCMTSTDIDDDMNWYQQKPGQAPRLLISEGNTLRPGVPARFSGSGSGTDFTLTISS
LEPEDFAVYYCLQRAGYPVTFGQGTKVEIKRGGGGSGGGGSGGGGSEVQLVESGGGLVQPGGSLRLSCVTSGFTFRK
FGMSWVRQAPGKGLEWVATIAT-GGHTTDYSDSVKGRFTISRDNSKNTLYLQMNSLRAEDTAVYYCTRA-G-KG-
AAFAYWGQGTLVTVSS
>P3.12
DIVLTQSPATLSLSPGERATVSCMTSTDIDDDMNWYQQKPGQAPRLLISEGNTLRPGVPARFSGSGSGTDFTLTISS
LEPEDFAVYYCLQRSSYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-AG-
AGFAYWGQGTLVTVSS
>P3.11
DIVLTQSPATLSLSPGERATLSCMTSTDIDDDMNWYQQKPGQAPRLLISEGNTLRPGVPARFSGSGSGTDFTLTISS
LEPEDFAVYYCLQRSAYPITFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRA-G-AG-
GAFAYWGQGTLVTVSS
>P3.10
DIVLTQSPATLSLSPGERATLSCMTSTDIDDDMNWYQQKPGQAPRLLISEGNTLRPGVPARFSGSGSGTDFTLTISS
LEPEDFAVYYCLQRTGYPITFGQGTKVEIKRGGGGSGGGGSGGGGSEVQLVESGGGLVQPGGSLRLSCVTSGFTFRK
FGMSWVRQAPGKGLEWVATISTPRGSSTNYSDSVKGRFTISRDNSKNTLYLQMNSLRAEDTAVYYCTRA-G-DG-
AAFDYWGQGTLVTVSS
>P3.6
DIVLTQSPATLSLSPGERATLSCMTSTDIDDDMNWYQQKPGQAPRLLISEGNTLRPGVPARFSGSGSGTDFTLTISS
LEPEDFAVYYCLQRAGFPLTFGQGTKVEIKRGGGGSGGGGSGGGGSEVQLVESGGGLVQPGGSLRLSCVTSGFTFRK
FGMSWVRQAPGKGLEWVATIAT-GGHTTNYSDSVKGRFTISRDNSKNTLYLQMNSLRAEDTAVYYCTRA-G-
AGGAAFDYWGQGTLVTVSS
>P3.5
DIVLTQSPATLSLSPGERATLSCMTSTDIDDDMNWYQQKPGQAPRLLISEGNTLRPGVPARFSGSGSGTDFTLTISS
LEPEDFAVYYCLQRSAFPLTFGQGTKVEIKRGGGGSGGGGSGGGGSEVQLVESGGGLVQPGGSLRLSCVTSGFTFRK
FGMSWVRQAPGKGLEWVASISTPRGSDTDYSDSVKGRFTISRDNSKNTLYLQMNSLRAEDTAVYYCTRA-G-AG-
SSFDYWGQGTLVTVSS
>P3.4
DIVLTQSPATLSLSPGERATLSCMTSTDIDDDMNWYQQKPGQAPRLLISEGNTLRPGVPARFSGSGSGTDFTLTISS
LEPEDFAVYYCLQRATYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-AG-
TAFDYWGQGTLVTVSS
>P3.1
DIVLTQSPATLSLSPGERATLSCMTSTDIDDDMNWYQQKPGQAPRLLISEGNTLRPGVPARFSGSGSGTDFTLTISS
LEPEDFAVYYCLQRSAFPITFGQGTKVEIKRGGGGSGGGGSGGGGSEVQLVESGGGLVQPGGSLRLSCVTSGFTFRK
FGMSWVRQAPGKGLEWVATISTPRGSTTDYSDSVKGRFTISRDNSKNTLYLQMNSLRAEDTAVYYCTRT-A-AG-
AAFAYWGQGTLVTVSS
>P3.28
DIVLTQSPATLSLSPGERATLSCMTSTDIDDDMNWYQQKPGQAPRLLISEGNTLRPGVPARFSGSGSGTDFTLTISS
LEPEDFAVYYCLQRNAYPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRA-G-NG-
AAMAYWGQGTLVTVSS
>P3.19
DIVLTQSPATLSLSPGERATVSCMTSTDIDDDMNWYQQKPGQAPRLLISEGNTLRPGVPARFSGSGSGTDFTLTISS
LEPEDFAVYYCLQHYSFPITFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTRGAR-RG-
EGFDYWGQGTLVTVSS
>P3.8
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTRGKG----
KKMDYWGQGTLVTVSS
>P3.2
DIVLTQSPATLSLSPGERATLSCMTSTDIDDDMNWYQQKPGQAPRLLISEGNTLRPGVPARFSGSGSGTDFTLTISS
LEPEDFAVYYCLQRSAYPITFGQGTKVEIKRGGGGSGGGGSGGGGSEVQLVESGGGLVQPGGSLRLSCVTSGFTFRK
FGMSWVRQAPGKGLEWVASIAT-GGHTTNYSDSVKGRFTISRDNSKNTLYLQMNSLRAEDTAVYYCTRA-G-GG-
ASFAYWGQGTLVTVSS
>P4.B9
DIVLTQSPATLSLSPGERATLSCMTSTDIDDDMNWYQQKPGQAPRLLISEGNTLRPGVPARFSGSGSGTDFTLTIS
SLEPEDFAVYYCLQGTNDPVTFGQGTKVEIKRGGGGSGGGGSGGGGSEVQLVESGGGLVQPGGSLRLSCVTSGFTF
RKFGMSWVRQAPGKGLEWVASISTPRGSSTYYSDSVKGRFTISRDNSKNTLYLQMNSLRAEDTAVYYCTRGYSSTS
YAMDYWGQGTLVTVSS
>P4.A11
DIVLTQSPATLSLSPGERATLSCMTSTDIDDDMNWYQQKPGQAPRLLISEGNTLRPGVPARFSGSGSGTDFTLTIS
SLEPEDFAVYYCLQRNAVPFTFGQGTKVEIKRGGGGSGGGGSGGGGSEVQLVESGGGLVQPGGSLRLSCVTSGFTF
RKFGMSWVRQAPGKGLEWVAGISTPRGSYTDYSDSVKGRFTISRDNSKNTLYLQMNSLRAEDTAVYYCTRG-GGAG
AGFDYWGQGTLVTVSS
>P5.G5
DIVLTQSPATLSLSPGERATLSCMTSTDIDDDMNWYQQKPGQAPRLLISEGNTLRPGVPARFSGSGSGTDFTLTISS
LEPEDFAVYYCLQHYDIPLTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------GKA--
MDYWGQGTLVTVSS
>P5.H11
DIVLTQSPATLSLSPGERATLSCMTSTDIDDDMNWYQQKPGQAPRLLISEGNTLRPGVPARFSGSGSGTDFTLTISS
LEPEDFAVYYCLQSDSFPVTFGQGTKVEIKRGGGGSGGGGSGGGGSEVQLVESGGGLVQPGGSLRLSCVTSGFTFRK
FGMSWVRQAPGKGLEWVATIATGGHTTYYSDSVKGRFTISRDNSKNTLYLQMNSLRAEDTAVYYCTRWSWDNRSAT--
MDYWGQGTLVTVSS
>P5.G10
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------
STAAWFDYWGQGTLVTVSS
>P5.C5
DIVLTQSPATLSLSPGERATLSCMTSTDIDDDMNWYQQKPGQAPRLLISEGNTLRPGVPARFSGSGSGTDFTLTISS
LEPEDFAVYYCLQYDAFPFTFGQGTKVEIKRGGGGSGGGGSGGGGSEVQLVESGGGLVQPGGSLRLSCVTSGFTFRK
FGMSWVRQAPGKGLEWVASIATGGHTTYYSDSVKGRFTISRDNSKNTLYLQMNSLRAEDTAVYYCTR------GGE-
MDYWGQGTLVTVSS
>P6.G11
DIVLTQSPATLSLSPGERATLSCMTSTDIDDDMNWYQQKPGQAPRLLISEGNTLRPGVPARFSGSGSGTDFTLTISS
LEPEDFAVYYCLQYSDLPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
E--G---FAYWGQGTLVTVSS
>P6.G10
DIVLTQSPATLSLSPGERATLSCMTSTDIDDDMNWYQQKPGQAPRLLISEGNTLRPGVPARFSGSGSGTDFTLTISS
LEPEDFAVYYCLQDASFPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----ATTT
S--T-----FAYWGQGTLVTVSS
>P6.G8
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----A---
E--N-----MDYWGQGTLVTVSS
>P6.G7
DIVLTQSPATLSLSPGERATLSCMTSTDIDDDMNWYQQKPGQAPRLLISEGNTLRPGVPARFSGSGSGTDFTLTISS
LEPEDFAVYYCLQYNGIPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
T--G-----FDYWGQGTLVTVSS
>P6.G6
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
A--S-----MDYWGQGTLVTVSS
>P6.G5
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
E--S-----MDYWGQGTLVTVSS
>P6.G4
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
GYYA-----FDYWGQGTLVTVSS
>P6.F12
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
A--S-----MDYWGQGTLVTVSS
>P6.F11
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASISTPRGSTTYYSDSVKGRFTISRDNSKNTLYLQMNSLRAEDTAVYYCTR-----T---
N--N--SSWFDYWGQGTLVTVSS
>P6.F3
DIVLTQSPATLSLSPGERATLSCMTSTDIDDDMNWYQQKPGQAPRLLISEGNTLRPGVPARFSGSGSGTDFTLTISS
LEPEDFAVYYCLQYAGVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
S--A-----MDYWGQGTLVTVYS
>P6.F2
DIVLTQSPATLSLSPGERATLSCMTSTDIDDDMNWYQQKPGQAPRLLISEGNTLRPGVPARFSGSGSGTDFTLTISS
LEPEDFAVYYCLQYAGVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----N---
A--T-----MDYWGQGTLVTVSS
>P6.F1
DIVLTQSPATLSLSPGERATLSCMTSTDIDDDMNWYQQKPGQAPRLLISEGNTLRPGVPARFSGSGSGTDFTLTISS
LEPEDFAVYYCLQGDAIPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----D---
S--G-----MDYWGQGTLVTVSS
>P6.E12
DIVLTQSPATLSLSPGERATLSCMTSTDIDDDMNWYQQKPGQAPRLLISEGNTLRPGVPARFSGSGSGTDFTLTISS
LEPEDFAVYYCLQHYNVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
Y--G-----FDYWGQGTLVTVSS
>P6.E11
DIVLTQSPATLSLSPGERATLSCMTSTDIDDDMNWYQQKPGQAPRLLISEGNTLRPGVPARFSGSGSGTDFTLTISS
LEPEDFAVYYCLQYADIPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
G--S-----MDYWGQGTLVTVSS
>P6.E8
DIVLTQSPATLSLSPGERATLSCMTSTDIDDDMNWYQQKPGQAPRLLISEGNTLRPGVPARFSGSGSGTDFTLTISS
MEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
S--T-----FDYWGQGTLVTVSS
>P6.E7
DIVLTQSPATLSLSPGERATLSCMTSTDIDDDMNWYQQKPGQAPRLLISEGNTLRPGVPARFSGSGSGTDFTLTISS
LEPEDFAVYYCLQYTSVPFTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----A---
D--E-----MDYWGQGTLVTVSS
>P6.E6
DIVLTQSPATLSLSPGERATLSCMTSTDIDDDMNWYQQKPGQAPRLLISEGNTLRPGVPARFSGSGSGTDFTLTISS
LEPEDFGVYYCLQDYGFPVTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----S---
S--N--KDWFDYWGQGTLVTVSS
>P6.E2
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----K---
S--T-----FDYWGQGTLVTVSS
>P6.D6
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVAAIAT-GGHTTYYSDSVKGRFTISRDNSKNTLYLQMNSLRAEDTAVYYCTRSYYS-----
G--S-----MDYWGQGTLVTVSS
>P6.C12
DIVLTQSPATLSLSPGERATLSCMTSTDIDDDMNWYQQKPGQAPRLLISEGNTLRPGVPARFSGSGSGTDFTLTISS
LEPEDFAVYYCLQSFNVPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----R---
A--N-----FDYWGQGTLVTVSS
>P6.C6
DIVLTQSPATLSLSPGERATLSCMTSTDIDDDMNWYQQKPGQAPRLLISEGNTLRPGVPARFSGSGSGTDFTLTISS
LEPEDFAVYYCLQSASIPLTFGQGTKVEIKRGGGGSGGGGSGGGGSEVQLVESGGGLVQPGGSLRLSCVTSGFTFRK
FGMSWVRQAPGKGLEWVASIAT-GGHTTYYSDSVKGRFTISRDNSKNTLYLQMNSLRAEDTAVYYCTR-----G---
E--A-----MDYWGQGTLVTVSS
>P10.G12
DIVLTQSPATLSLSPGERATLSCMTSTDIDDDMNWYQQKPGQAPRLLISEGNTLRPGVPARFSGSGSGTDFTLTIS
SLEPEDFAVYYCLQYAGLPLTFGQGTKVEIKRGGGGSGGGGSGGGGSEVQLVESGGGLVQPGGSLRLSCVTSGFTF
RKFGMSWVRQAPGKGLEWVAGISTPRGSNTYYSDSVKGRFTISRDNSKNTLYLQMNSLRAEDTAVYYCTRAGRGEG-
FAYWGQGTLVTVSS
>P10.D6
DIVLTQSPATLSLSPGERATLSCMTSTDIDDDMNWYQQKPGQAPRLLISEGNTLRPGVPARFSGSGSGTDFTLTIS
SLEPEDFAVYYCLQHATVPFTFGQGTKVEIKRGGGGSGGGGSGGGGSEVQLVESGGGLVQPGGSLRLSCVTSGFTF
RKFGMSWVRQAPGKGLEWVASISTPRGSTTYYSDSVKGRFTISRDNSKNTLYLQMNSLRAEDTAVYYCTRTT-WNN
YFAYWGQGTLVTVSS
>P10.B10
DIVLTQSPATLSLSPGERATLSCMTSTDIDDDMNWYQQKPGQAPRLLISEGNTLRPGVPARFSGSGSGTDFTLTIS
SLEPEDFAVYYCLQHNTFPFTFGQGTKVEIKRGGGGSGGGGSGGGGSEVQLVESGGGLVQPGGSLRLSCVTSGFTF
RKFGMSWVRQAPGKGLEWVARISTPRGSNTDYSDSVKGRFTISRDNSKNTLYLQMNSLRAEDTAVYYCTRGG-AYN-
FAYWGQGTLVTVSS
>P10.H2
DIVLTQSPATLSLSPGERATLSCMTSTDIDDDMNWYQQKPGQAPRLLISEGNTLRPGVPARFSGSGSGTDFTLTIS
SLEPEDFAVYYCLQGSGAPFTFGQGTKVEIKRGGGGSGGGGSGGGGSEVQLVESGGGLVQPGGSLRLSCVTSGFTF
RKFGMSWVRQAPGKGLEWVAEIAT-GGHTTYYSDSVKGRFTISRDNSKNTLYLQMNSLRAEDTAVYYCTRGG-SMD---
YWGQGTLVTVSS
>pSAb_HC (Heavy Chain)
EISEVQLVESGGGLVQPGGSLRLSCVTSGFTFRKFGMSWVRQAPGKGLEWVASIAT-GGHTTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pSTAb_HC (Heavy Chain)
EISEVQLVESGGGLVQPGGSLRLSCVTSGFTFRKFGMSWVRQAPGKGLEWVASISTPRGSTTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pYAb_HC (Heavy Chain)
EISEVQLVESGGGLVQPGGSLRLSCVTSGFTFRKFGMSWVRQAPGKGLEWVASIVG--GRKTYYSDSVKGRFTISR
DNSKNTLYLQMNSLRAEDTAVYYCTRGYSSTSYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCD
KTHT
>pSAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
>pSTAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
>pYAb_LC
DIVLTQSPATLSLSPGERATLSCMTSTDIDDDMNWYQQKPGQAPRLLISEGNTLRPGVPARFSGSGSGTDFTLTIS
SLEPEDFAVYYCLQSFNVPLTFGQGTKVEIKRTVAAPSVFIFPPSDSQLKSGTASVVCLLNNFYPREAKVQWKVDN
ALQSGNSQESVTEQDSKDSTYSLSSTLTLSKADYEKHKVYACEVTHQGLSSPVTKSFNRGEC
ID P8.H9 STANDARD;
PRT; 241 AA
SQ Sequence 241 AA;

| | |
|---|---|
| DIVLTQSPAT LSLSPGERAT LSCMTSTDID DDMNWYQQKP GQAPRLLISE GNTLRPGVPA | 60 |
| RFSGSGSGTD FTLTISSLEP EDFAVYYCLQ STGVPFTFGQ GTKVEIKRGG GGSGGGGSGG | 120 |
| GGSEVQLVES GGGLVQPGGS LRLSCVTSGF TFRKFGMSWV RQAPGKGLEW VASIATGGHT | 180 |
| TYYSDSVKGR FTISRDNSKN TLYLQMNSLR AEDTAVYYCT RNSSDAWMAY WGQGTLVTVS | 240 |
| S | 241 |

What is claimed is:

1. A method of identifying antibodies that specifically bind a protein comprising a post translational modification, the method comprising the steps of:
(1) identifying a noncovalent post translational modification-binding motif in a protein;
(2) inspecting known antibody CDRs for an anchoring pocket sequence that adopts the conformation of the noncovalent post translational modification-binding motif;
(3) preparing an antibody scaffold comprising a CDR, wherein amino acid residues from the CDR comprise the anchoring pocket;
(4) preparing a library comprising the antibody scaffold, wherein the CDR is randomized outside of the anchoring pocket; and
(5) identifying antibodies in the library that specifically bind to a protein comprising a post translational modification.

2. The method of claim 1, wherein the antibody binds a specific protein comprising the post translational modification.

3. The method of claim 1, wherein the antibody binds a specific post translational modification.

4. The method of claim 3, wherein the post translational modification is an anion.

5. The method of claim 3, wherein the post translational modification is phosphorylation, sulfation, acetylation, S-nitrosylation, methylation, proteolysis, or glycosylation.

6. The method of claim 1, wherein the noncovalent post translational modification-binding motif recognizes an anion.

7. The method of claim 1, wherein the noncovalent post translational modification-binding motif recognizes a phosphate, a sulfate, an acetyl, a methyl, a nitric oxide, a N-terminal alpha amine, a C-terminal carboxylate, a GalNAc sugar or a GlcNAc sugar.

8. The method of claim 1, further comprising the step of characterizing the anchoring pocket.

9. The method of claim 1, wherein amino acid residues from two or more CDRs comprise the anchoring pocket.

10. A method of identifying antibodies that specifically bind a protein comprising a post translational modification, the method comprising the steps of:
(1) identifying a noncovalent post translational modification-binding motif in a protein;
(2) engineering an antibody scaffold comprising a CDR, wherein amino acid residues from the CDR comprise an anchoring pocket that adopts the conformation of the noncovalent post translational modification-binding motif;
(3) preparing a library comprising the antibody scaffold, wherein the CDR is randomized outside of the anchoring pocket; and
(4) identifying antibodies in the library that specifically bind to a protein comprising a post translational modification.

11. The method of claim 10, wherein the antibody binds a specific protein comprising the post translational modification.

12. The method of claim 10, wherein the antibody binds a specific post translational modification.

13. The method of claim 12, wherein the post translational modification is an anion.

14. The method of claim 12, wherein the post translational modification is phosphorylation, sulfation, acetylation, S-nitrosylation, methylation, proteolysis, or glycosylation.

15. The method of claim 10, wherein the noncovalent post translational modification-binding motif recognizes an anion.

16. The method of claim 10, wherein the noncovalent post translational modification-binding motif recognizes a phosphate, a sulfate, an acetyl, a methyl, a nitric oxide, a N-terminal alpha amine, a C-terminal carboxylate, a GalNAc sugar or a GlcNAc sugar.

17. The method of claim 10, further comprising the step of characterizing the anchoring pocket.

18. The method of claim 10, wherein amino acid residues from two or more CDRs comprise the anchoring pocket.
